(19) United States
(12) Patent Application Publication
(10) Pub. No.: US 2016/0251671 A1
(43) Pub. Date: Sep. 1, 2016

(54) TRANSGENIC TREES HAVING REDUCED XYLAN CONTENT

(71) Applicant: SWETREE TECHNOLOGIES AB, Umeå (SE)

(72) Inventors: Ewa J. MELLEROWICZ, Umeå (SE); Leif JÖNSSON, Umeå (SE); Christine RATKE, Umeå (SE); Barbara TEREBIENIEC, Umeå (SE); Sandra WINESTRAND, Karlstad (SE)

(21) Appl. No.: 15/030,686

(22) PCT Filed: Oct. 21, 2014
(86) PCT No.: PCT/SE2014/051238

§ 371 Date: Apr. 20, 2016

(30) Foreign Application Priority Data
Oct. 21, 2013 (SE) 1351248-8

(51) Int. Cl. C12N 15/82; C12N 15/113

(52) U.S. Cl. CPC C12N 15/8246; C12N 15/8218; C12N 15/1137; C12Y 204/01; C12N 2310/14

(57) ABSTRACT

The invention relates to a method for producing a transgenic tree having reduced xylan content compared to a wild-type tree of the same species, said method comprising reducing expression of one or more genes from the glycosyltransferase 43 (GT43), family in the said transgenic tree, whereby growth properties, mechanical properties, and/or saccharification properties are improved in the said transgenic tree.

### FIELD OF THE INVENTION

The invention relates to the field of improved properties in trees. Particularly the invention is related to a method of down regulating glycosyltransferase 43 genes in the xylan biosynthetic machinery with increased growth, improved saccharification and mechanical properties. The invention also relates to the used expression vector, the transgenic tree and the wood. Such trees and wood are useful in forest plantations as well as for bioenergy use.

### BACKGROUND TO THE INVENTION

Wood is an excellent energy source as an abundant raw material for renewable biofuels. The cell walls of higher plants contain polysaccharides such as cellulose, hemicelluloses, pectins and polyphenol compounds such as lignin. All of these cell wall components make up lignocellulosic biomass, which is a rich source of energy.

Xylan is the main hemicellulose in hardwoods, constituting approx. 20% of woody biomass. The length of the xylan backbone in hardwoods is usually within a narrow range of length for a species, but can vary according to the isolation procedure. The number of residues varies between 50 and 250. Presumably, the length of xylan is tightly controlled in hardwoods.

Xylans are synthesized by a number of glycosyltransferases (GT) in the Golgi and are transported in vesicles to the plasma membrane, where they become integrated into the cell wall and cross-linked with other wall compositions. In Populus the glycosyltransferase enzymes belongs to the gene families GT8, GT43 and GT47. They are required for initiation, elongation and termination of xylan backbone.

Biosynthesis of xylan is a field of intense studies since the first genes involved in xylan biosynthesis were discovered in Arabidopsis.

The Populus trichocarpa GT43 group includes seven genes designated GT43 A to G. The nucleic acid sequences are shown as SEQ ID NOS: 1 to 7; and the corresponding amino acid sequences are shown as SEQ ID NOS: 8 to 14. A comparative sequence analysis in Populus trichocarpa, Arabidopsis thaliana and Oryza sativa indicated that GT43 genes form three distinct clades that are conserved in three divergent genera, see FIG. 1. The Arabidopsis thaliana nucleic acid sequences are shown in the sequence listing as SEQ ID NO: 15 (AtlRX9; clade I); SEQ ID NO: 16 (AtlRX9-L; clade II); SEQ ID NO: 17 (AtlRX14; clade III) and SEQ ID NO: 18 (AtlRX14-L; clade III). The corresponding amino acid sequences are shown as SEQ ID NOS: 19 to 22, respectively.

Lower vascular plants and spruce have only two clades, clade II and clade III.

Studies have shown that a member of clade I or II and a member of clade III are required for xylan xylosyltransferase activity. This is supported by a recent study (Lee et al. (2012) Plant Signaling & Behavior 7: 1-6) which demonstrated that two poplar glycosyltransferases, PtrGT43B and PtrGT43C, are involved in xylan biosynthesis. Their findings indicate that poplar GT43 members act cooperatively in catalyzing the successive transfer of xylosyl residues during xylan backbone biosynthesis. This further supports the hypothesis that the biochemical functions of GT43 gene family members in vascular plants are evolutionarily conserved.

Populus GT43 members have been found to be involved in elongation of the xylan backbone. GT43A and B are highly expressed during wood formation, specifically expressed in cells undergoing secondary cell wall thickening.

The expression of the Populus GT43B gene has been reduced in transgenic 30 Populus by the RNAi (Lee et al. (2011) Mol. Plant 4: 730-747). This reduced expression lead to a reduction of these Populus transcripts to 2%-15% of WT level. As a consequence, abnormal xylem was formed with thin cell walls in fibers and vessels, and irregular xylem phenotype (collapsed vessels).

Furthermore, the xylose content was reduced to 50%-80% of the WT xylose content in these lines. S and G-lignin content was also reduced.

Pentoses, such as xylan, are considered as a poor substrate for fermentation, and therefore the reduction of xylan content would be beneficial in fermentation applications. However, plants with reduced xylan content are dwarf and/or produce xylem with collapsed and thin cell walls. Such plants are also weak and have brittle stems.

In the case when the GT43B gene expression was reduced by RNAi the lignin content was reduced (Lee et al. (2011) Mol. Plant 4: 730-747). Cellulose digestibility was increased in GT43B RNAi, GT47C RNAi lines compared to WT when same cellulose amount was subjected to enzymatic digestion. No information on saccharification yields per dry weight of lignocellulose was provided. Some poplar lines with xylan defects are weak and have brittle stems (Li et al. (2011) Tree Physiol. 31: 226-236). The growth of the trees was not affected in the case of GT47C suppression and it was not reported in the case of other transgenic lines.

WOs2012/103555 describes down-regulation of the IRX9 gene in Arabidopsis by antisense leading to reduced xylan content with reduced growth properties and reduced mechanical properties.

US 2012/0185975 discloses an isolated promoter from an Arabidopsis glycosyltransferase gene. It is discussed that it can be used in plant, plant part or plant cell for stress treatment.

A reduction of xylan content will lead to an increased proportion of cellulose in wood. With the knowledge that sugar derived from xylan is considered as a poor substrate for fermentation in contrast to sugar derived from cellulose, the reduction of xylan content would be beneficial. The problems with trees which have a reduced xylan content are, based on the discussion above, that they are dwarf and/or produce xylem with collapsed and/or thin cell walls. Such trees are also weak and have brittle stems.

Thus there is a need for a method and tools to make transgenic trees having increased growth with maintained or improved mechanical properties.

Furthermore, there is a need for better saccharification properties. Transgenic trees of this kind constitute a valuable material for forest plantations for general use and for bioenergy crops in particular.

### SUMMARY OF THE INVENTION

In a first aspect, the invention provides a method for producing a transgenic tree having reduced xylan content compared to a wild-type tree of the same species, said method comprising reducing expression of one or more genes from the glycosyltransferase 43 (GT43) family in the said transgenic tree, wherein said transgenic tree have increased growth properties and improved mechanical properties, and/or saccharification properties compared to a wild-type tree of the same species.

In one embodiment, the method according to this aspect comprises the steps of:
(a) introducing an expression vector into a tree cell, said expression vector comprising:
   (i) at least one promoter
   (ii) a first nucleotide sequence comprising one or more genes from the GT43 family operably linked downstream of said promoter; and
   (iii) a second nucleotide sequence which is complementary to the first nucleotide sequence operably linked downstream of said first nucleotide sequence;
   wherein the said linked nucleotide sequences are transcribed into RNA in the cell, thereby producing a hairpin RNA which is processed in the cell to an interfering RNA molecule capable of reducing expression of one or more genes from the GT43 family; and
(b) culturing said tree cells of step (a) to a transgenic tree and vegetatively propagating of the said transgenic tree to obtain a transgenic tree line.

The transgenic tree lines having the expression of GT43 genes reduced specifically in the developing wood are selected from a large number of lines obtained, preferably no less than from 20 lines.

In a preferred embodiment the said promoter is a wood-specific promoter which is up-regulated during secondary wall biosynthesis.

In another preferred embodiment the said wood-specific promoter is selected from a GT43 promoter, secondary wall CesA promoters, RWA promoters, especially promoters homologous to Arabidopsis RWA1, RWA3 or RWA4 promoters, promoters homologous to Arabidopsis IRX10 promoter, GUX1 or GUX 2 promoters, AtFRA8, AtlRX8/or AtPARVUS promoters, AtXyn1 promoter, AtTBL29 promoter. It is known in the art that these promoters are predominantly expressed in vascular tissue and are involved in secondary wall formation and their expression patterns are easy to follow using data from wood development.

In yet another preferred embodiment, the said wood-specific promoter is a GT43B promoter comprising the nucleotide sequence shown as SEQ ID NO: 25 or a GT43B-derived wood-specific (WP) promoter comprising the nucleotide sequence shown as SEQ ID NO: 23. The difference between the GT43B promoter and the WP promoter is deletion of 24 nucleotides up-stream of the starting codon.

According to another embodiment, wherein in developing wood of a said transgenic tree, the level of mRNA transcribed from the said one or more genes from the GT43 family is between 25% and 85% of the corresponding levels of mRNA transcribed in wild-type tree of the same species.

Preferably, in developing wood of a said transgenic tree, the level of mRNA transcribed from the said one or more genes from the GT43 family, preferably GT43A or GT43B or GT43C, is reduced but not abolished. The said mRNA level is preferably between 25% and 85%, between 35% and 75%, between 30% and 60%, or more preferably between 50% and 60%, of the corresponding levels of mRNA transcribed in wild-type tree of the same species. The level of the suppression may be targeted to developing wood tissues by using wood-specific promoters, i.e. a promoter predominantly expressed in vascular tissue. The said reduced expression is preferably achieved by an RNA interference (RNAi) process which is well known in the art.

The said one or more genes from the GT43 family are preferably selected from
(a) the group of Populus trichocarpa genes consisting of:
   GT43A (SEQ ID NO: 1),
   GT43B (SEQ ID NO: 2),
   GT43C (SEQ ID NO: 3),
   GT43D (SEQ ID NO: 4),
   GT43E (SEQ ID NO: 5),
   GT43F (SEQ ID NO: 6),
   GT43G (SEQ ID NO: 7);
(b) genes which are orthologous to the Populus trichocarpa genes in (a) and which have at least 80% sequence identity with the genes in (a).

The said orthologous genes in (b) are preferably from a species selected from Eucalyptus, Acacia, or Salix.

The said Eucalyptus genes are selected from the group consisting of:
GT43A (SEQ ID NO: 31),
GT43B (SEQ ID NO: 32),
GT43C (SEQ ID NO: 33),
GT43D (SEQ ID NO: 34),
GT43E (SEQ ID NO: 35),
GT43F (SEQ ID NO: 36),
GT43G (SEQ ID NO: 37).

Preferably, the said one or more genes from the GT43 family are selected from the group consisting of GT43B (SEQ ID NO: 2), GT43C (SEQ ID NO: 3), and GT43E (SEQ ID NO: 5). More preferably, the genes are selected from the group consisting of GT43B (SEQ ID NO: 2), and GT43C (SEQ ID NO: 3). Even more preferably, the method according to the invention comprises reducing expression of both GT43B and GT43C or reducing expression of the three GT43 genes GT43B, GT43C and GT43E.

As mentioned above, one embodiment of the method of the invention relates to the production of a transgenic tree having increased growth. In this context the term “increased growth” means one or more features selected from increased height, increased stem diameter, increased stem volume, increased internode length, and increased number of leaves.

Further, one embodiment of the method of the invention relates to the production of a transgenic tree having improved mechanical properties. In this context the term “improved mechanical properties” means increased modulus of elasticity (MOE) and/or increased bending strength (BS).

In yet another embodiment, the method of the invention provides the production of a transgenic tree, from which the yield of monosaccharides from hydrolyzed wood is increased compared to wood from a wild-type tree of the same species. The said monosaccharides are preferably selected from the group consisting of arabinose, galactose, glucose, xylose and mannose.

A second aspect of the invention relates to a transgenic tree obtainable by the method as disclosed above in the first aspect. The said transgenic tree is preferably of a genus selected from Populus, Eucalyptus, Acacia or Salix. Further, the invention relates to a transgenic tree comprising the expression cassette (or vector) of the fourth aspect of the invention

In a third aspect, the invention also relates to wood obtainable from the transgenic tree according to the second aspect of the invention.

In the wood of the said transgenic tree, the level of the mRNA of one or more genes from the GT43 is reduced when compared to wood from a wild-type tree of the same species. In particular, the said level of mRNA is reduced but not abolished. Preferably, the level of the mRNA is reduced to between 25% and 85%, more preferably between 30% and 60% of the mRNA when compared to a wild-type tree of the same species.

A fourth aspect of the invention relates to an expression vector comprising:
(i) a wood-specific promoter which is active in developing wood;
(ii) at least one gene or a part of a gene having at least 18 nucleotides, optionally selected from the glycosyltransferase 43 (GT43) gene family.

In one embodiment of this aspect, the invention relates to an expression vector comprising:
(i) at least one promoter;
(ii) a first nucleotide sequence comprising one or more genes from the GT43 family; and
(iii) a second nucleotide sequence which is complementary to the first nucleotide sequence;
   wherein the said nucleotide sequences are capable of being transcribed in a tree cell into a hairpin RNA structure which is processed in the cell to an interfering RNA molecule capable of reducing expression of a GT43 gene.

As discussed above, the promoter is a promoter predominantly expressed in developing xylem, such as a GT43B promoter. The said expression vector could further comprise one or more regulatory elements selected from the group consisting of transcriptor factor binding sites, splice sites and termination sites.

The said one or more genes from the GT43 family are preferably selected from the group consisting of GT43B (SEQ ID NO: 2), and GT43C (SEQ ID NO: 3). In one preferred embodiment, the said first nucleotide sequence comprises two or three genes from the GT43 family. Preferably the said two or three genes include both GT43B (SEQ ID NO: 2) and GT43C (SEQ ID NO: 3).

The said one or more genes from the GT43 family may preferably be selected from both GT43B and GT43C or the three genes GT43B, GT43C and GT43E.

In yet another aspect, the invention includes a method according to any one of the preceding aspects for producing a transgenic tree, from which the yield of monosaccharides from hydrolyzed wood is increased compared to wood from a wild-type tree of the same species. The said monosaccharides are preferably selected from the group consisting of arabinose, galactose, glucose, xylose and mannose.

A further aspect of the invention relates to the use of the expression vector of the invention for increased expression or for down-regulation of a gene, preferably a gene selected from the glycosyltransferase 43 (GT43) gene family.

In yet another aspect, the invention provides a method for producing monosaccharides from wood. The said method comprises (a) providing wood from a transgenic tree as described above; (b) degrading the wood; and (c) obtaining free monosaccharides. The wood is degraded by enzymes either with or without pre-treatment under acidic conditions. In the case of pre-treatment, acidic conditions can be created in many ways, using various acids and acid concentrations. E.g. in a preferred embodiment acidic conditions can be created by the addition of sulphuric acid at a concentration of 1% (weight/weight). Further, acidic conditions is in this context also intended to cover pretreatment methods where no external acid is added. Temperature elevation can be used as pretreatment in order to obtain acidic conditions. E.g. steam explosion without addition of acid is performed under acidic conditions because acids are formed during the pretreatment.

In an additional aspect, the invention relates to a process for using the monosaccharides that are obtained from wood according to the invention as substrates for microbial fermentation processes, as well as the products obtained by such a process. The products of such processes can be biofuels, other chemicals and biopolymers.

### BRIEF DESCRIPTION OF THE DRAWINGS

FIG. 1: Phylogenetic tree showing the members of the GT43 family in Populus trichocarpa (Pt), Arabidopsis thaliana (At) and Oryza sativa (Os) grouped into three clades (I-III).

FIG. 2: Maps of RNAi constructs targeting individual and multiple GT43, genes driven by the constitutive CaMV35S (35S) and the wood-specific (WP) promoters.

FIG. 3: Effects of the RNAi constructs on growth in the greenhouse. Different RNAi constructs (B, C, BE, BC, and BCE) are driven by either 35S or WP promoters, and each combination is represented by three independent lines. Significant effects compared to the WT are indicated by stars * (P≦5%, Tukey HSD test). Crossed lines indicate a significant difference between promoters FIG. 3A Height in % of wild type; FIG. 3B Number of leaves in % of wild type; FIG. 3C, Internode length in % of wild type; FIG. 3D Diameter in % of wild type; FIG. 3E Volume in % of wild type.

FIG. 4: Sugar yields in experiments without acid pretreatment. The figure shows the yields of monosaccharides after enzymatic hydrolysis.

FIG. 5: Sugar yields in experiment with acid-pretreated aspen. The FIG. 25 shows the monosaccharide yields after enzymatic hydrolysis combined with the monosaccharide yields in the pretreatment liquid.

FIG. 6: Plant growth of transgenic hybrid aspen containing the GT43B promoter driven RNAi construct GT43BC. A Plant height, stem diameter and stem volume. B Internode number and internode length. C Leaf length and width. A-C Data are means±SE, n=5 biol replicates. The star indicates values significantly different from WT (Student's t test, α=0.05).

FIG. 7: Expression levels of GT43 genes in transgenic hybrid aspen wood containing GT43 RNAi constructs. Relative expression to wild type (Y-axis)

FIG. 8: Transcript profiling: expression of xylan biosynthetic genes in transgenic hybrid aspen wood with reduced GT43 expression levels. Expression of genes involved in xylan backbone, glucuronic acid decorations and the reducing end oligosaccharide as well as xylan acetylation genes. Shown are fold changes of expressed genes in individual lines (BC1 and BC2) and both lines grouped compared to WT expression. Average pvalues (padj) are indicated beside the columns. Stars indicate p value below 0.1.

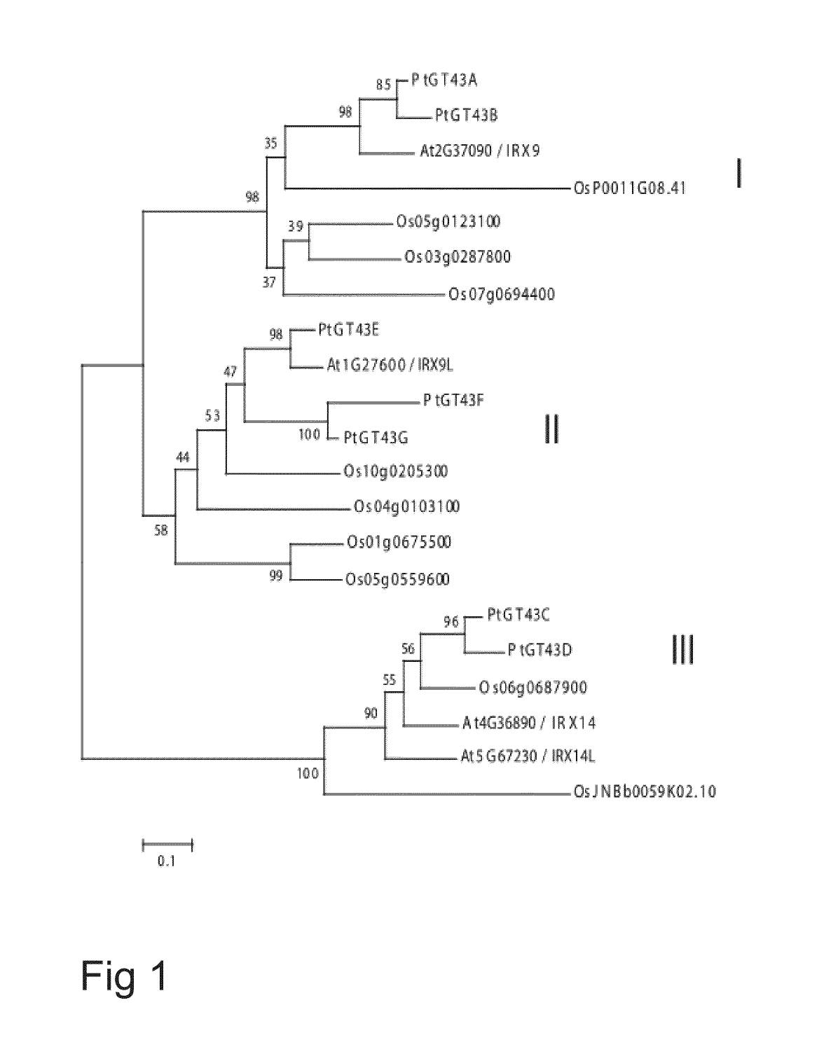

### DEFINITIONS

The phrase “sequence identity” in the context of two nucleic acids, may refer to two or more sequences or sub-sequences that have at least about 60%, 65%, 70%, 75%, preferably 80% or 85%, more preferably 90%, 91%, 92%, 93%, 94%, 95%, 96%, 97%, 98%, 99%, 99.5%, or greater, identity when compared and aligned for maximum correspondence, as measured using a sequence comparison algorithm or by visual inspection. The identity may refer to one gene, two or a group of genes separately and not to the other genes in the present invention, for example one gene might be 75% homologous and another might be 60% homologous and further another might be 93% homologous to the gene of interests. In certain aspects, substantial identity exists over a region of nucleic acid sequences of at least about 150 nucleic acid residues, such as at least about 200, 250, 300, 330, 360, 375, 400, 425, 450, 460, 480, 500, 600, 700, 800 such as at least about 900 nucleotides or such as at least about 1 kb, 2 kb, or such as at least about 3 kb. In some aspects, the nucleic acid sequences are identical over the entire length of the corresponding coding region.

The term “Reduced xylan content” refers to the amount of xylan that can be extracted from wood, it can be also related to the length of the xylan chain.

The term “wood-specific promoter” is a promoter predominantly expressed in vascular tissue.

Expression of a gene is the process when an mRNA is translated into a protein or enzyme Gene expression can be modulated at different levels from transcriptional initiation to RNA processing. By reducing the expression the stimuli by the protein or enzyme is expected to be reduced. In this context the term “reducing expression” is referred to lowering the RNA transcribed from a gene, which in most cases results in a lower amount of the mRNA. This can be the steady-state level of mRNA.

### DETAILED DESCRIPTION OF THE INVENTION

In a study of wood formation a series of transgenic hybrid aspen was made by reducing the expression of all genes in the GT43 family or combinations of two or three clades of these genes from the same family by the RNAi genetic approach. Then it was unexpectedly noticed that these transgenic trees had an increased growth and the wood had improved mechanical properties as seen in modulus of elasticity (MOE) and bending strength (BS) in three-point bending experiments contrary to what was seen earlier.

In a further test of the lignocellulose from wood from these transgenic hybrid aspen it was surprisingly noted that the lignocellulose of such trees exhibited better saccharification properties in tests without pre-treatment.

Identification of a Tissue Specific Promoter

Tissue-specific expression of transgenes at a high level is in most cases the best strategy for inducing desirable changes in the metabolism of development of that tissue.

There is a need for a tissue specific promoter that is specifically expressed in developing wood and is functional as good as or better than the frequently used cauliflower mosaic virus 35S promoter.

In order to identify a good promoter from the GT43 gene family, which is driving a high expression in developing wood, the transcript levels of all seven GT43 genes were quantified in vegetative aerial tissues by qRT-PCR, and the promoters from all the genes were cloned to drive the GUS gene for easy detection. The transgenic aspen lines were generated and analysed for GUS expression patterns.

The GUS activity for the three promoters pGT43A, pGT43B and pGT43C was found in developing wood and developing phloem fibers and expression patterns were more specific compared to the 35S promoter.

The sequences of all GT43 promoters are shown in the Sequence Listing. The last six positions in SEQ ID NO: 23 to 30 correspond to the two first amino acids in the protein.

Selection of GT43 Genes

The coding sequences of the genes GT43 A to G have different length in different trees. In poplar the length is between 666 (SEQ ID NO: 6) and 1533 (SEQ ID NO: 3) base pairs. In Eucalyptus the length is between 1008 (SEQ ID NO: 37) and 1509 (SEQ ID NO: 36) base pair. A fragment from any of these genes can be selected, the fragment can be from 20-50, 50-100, 100-200, 200-300, 300-400, 400-500, 500-600, 600-700, 800-900, 900-1000, 1000-1100, 1100-1200, 1200-1300, 1300-1400 or 1400-1500 bases long up to the full length of the coding sequence of the genes, even longer.

The GT43 genes are found in all plants synthesizing xylan. It is expected that the expressed proteins from these genes will have the same effect on the xylan synthesis.

The transcript levels of all seven GT43 genes were then quantified in by qRT-PCR in vegetative aerial tissues the transgenic hybrid aspen. The results are summarized in Table I, below.

TABLE I
Relative mRNA transcription level from qRT-PCR measurements in
different tissues of hybrid aspen

| | GT43A | GT43B | GT43C | GT43D | GT43E | GT43F | GT43G |
|---|---|---|---|---|---|---|---|
| Api- | 2% | 1% | 3% | 4% | 12% | 11% | 10% |
| cal | | | | | | | |
| shoot | | | | | | | |
| Leaf | 2% | 3% | 6% | 6% | 17% | 15% | 16% |
| Xy- | 61% | 66% | 52% | 50% | 16% | 34% | 40% |
| lem | | | | | | | |
| Ten- | 35% | 29% | 36% | 31% | 22% | 23% | 16% |
| sion | | | | | | | |
| wood | | | | | | | |
| Phlo- | 0% | 1% | 3% | 9% | 12% | 17% | 19% |
| em | | | | | | | |

Based on these data three GT43 genes were selected, one from each clade, on the basis of expression in the vegetative tissue, the xylem. The genes having high specificity and/or high expression level in the xylem are expected to have big impact on xylan biosynthesis in this tissue. For hybrid aspen GT43B (clade I), GT43E (clade II) and GT43C (clade III) were selected. Table II, below, show GT43 genes from Eucalyptus that may be used according to the invention.

TABLE II
Eucalyptus GT43 genes

| | | | Nucleic acid | Amino acid |
|---|---|---|---|---|
| | | Clade | sequence | sequence |
| GT43A | Eucgr.A01172.1 | I | SEQ ID NO: 31 | SEQ ID NO: 38 |
| GT43B | Eucgr.C00584.1 | II | SEQ ID NO: 32 | SEQ ID NO: 39 |
| GT43C | Eucgr.F00463.1 | II | SEQ ID NO: 33 | SEQ ID NO: 40 |
| GT43D | Eucgr.F02177.1 | II | SEQ ID NO: 34 | SEQ ID NO: 41 |
| GT43E | Eucgr.H02219.1 | III | SEQ ID NO: 35 | SEQ ID NO: 42 |
| GT43F | Eucgr.I00880.1 | III | SEQ ID NO: 36 | SEQ ID NO: 43 |
| GT43G | Eucgr.K03214.1 | I | SEQ ID NO: 37 | SEQ ID NO: 44 |

Generation of Transgenic Trees

Transgenic trees were produced using the genetic approach, interfering RNA to reduce or inhibit gene expression and explore gene function. RNAi constructs are usually driven by promoters in order to enhance and target the expression of the RNA which then will form the active RNAi hairpin. A commonly used promoter is the constitutive cauliflower 35S promoter.

All GT43 RNAi constructs used in this invention are driven by 35S or by the newly identified specific wood promoters. Any of the GT43 promoters can be used in the invention, but not excluding other promoters. The preferred promoter is the promoter derived from GT43B.

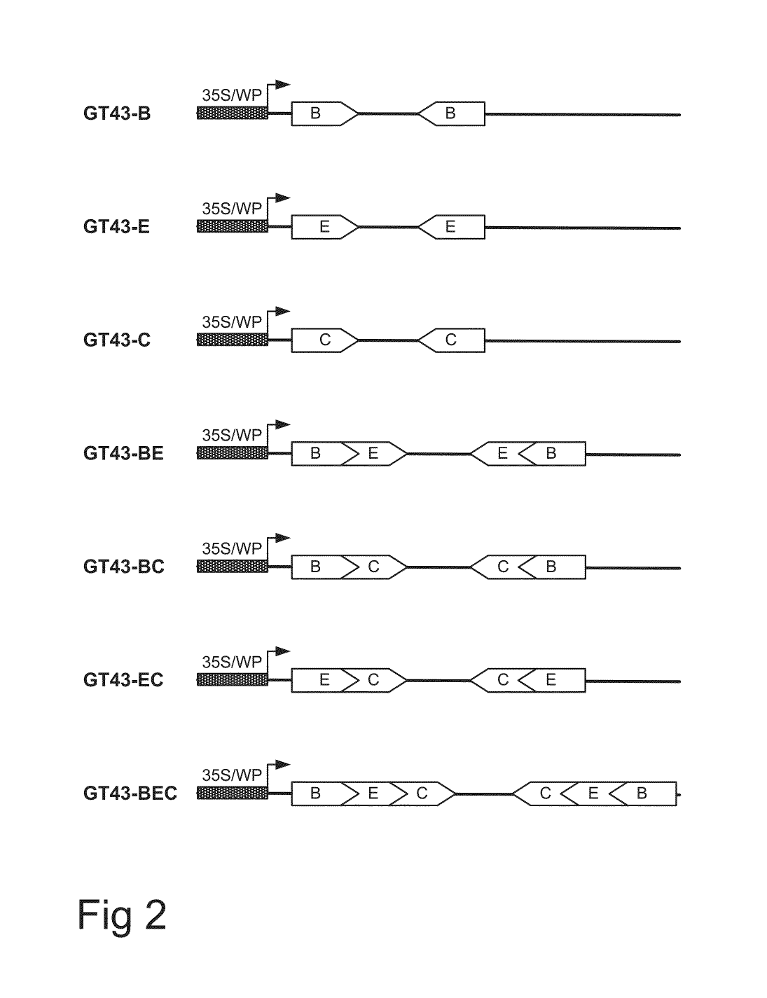

A set of different expression vectors, also called constructs, was genetically made. Each of them comprises a promoter, one, two or three fragments from the genes selected from the GT43 gene family. The same gene fragment was operationally cloned in the opposite direction forming an inverted repeat of the cloned gene fragment (FIG. 2).

Three of the GT43 genes; GT43B, SEQ ID NO: 2; GT43C, SEQ ID NO: 3; and GT43E, SEQ ID NO: 5 and their double (GT43BC, GT43BE, GT43CE) and triple (GT43BEC) combinations, were selected to be targeted by RNAi. All the constructs made were operable linked to the constitutive cauliflower 35S (35S) promoter or a wood specific (WP) promoter and transformed into hybrid aspen (Populus tremula×tremuloides).

These constructs were made to silence one clade, two clades, or all three clades at one time. All 7 types of inverted repeats were driven by either 35S or WP promoter, resulting in 14 constructs that were transferred to the hybrid aspen clone T89. These constructs are further described in the examples section below.

The RNA expressed in transgenic hybrid aspen lines transformed with these 14 constructs was studied and the transcript levels of GT43 genes were determined by qRT-PCR in samples from wood of 3 lines per construct, each with 3 biological replicates. The results (Table III) showed reduced transcription of GT43 genes by the different constructs to be between 35 and 75% of the wild-type level.

TABLE III
Expression of GT43 genes in wood of transgenic aspen lines

| | Constructs targeting: | | | | | | |
|---|---|---|---|---|---|---|---|
| | GT43B | GT43C | GT43E | GT43BE | GT43BC | GT43EC | GT43BCE |
| | Transcript levels of | | | | | | |
| | GT43B | GT43C | GT43E | GT43B | GT43B | GT43E | GT43B |
| WP | 36% | 70% | 55% | 41% | 55% | 71% | 64% |
| 35S | 55% | 50% | 66% | 43% | 43% | 57% | 75% |

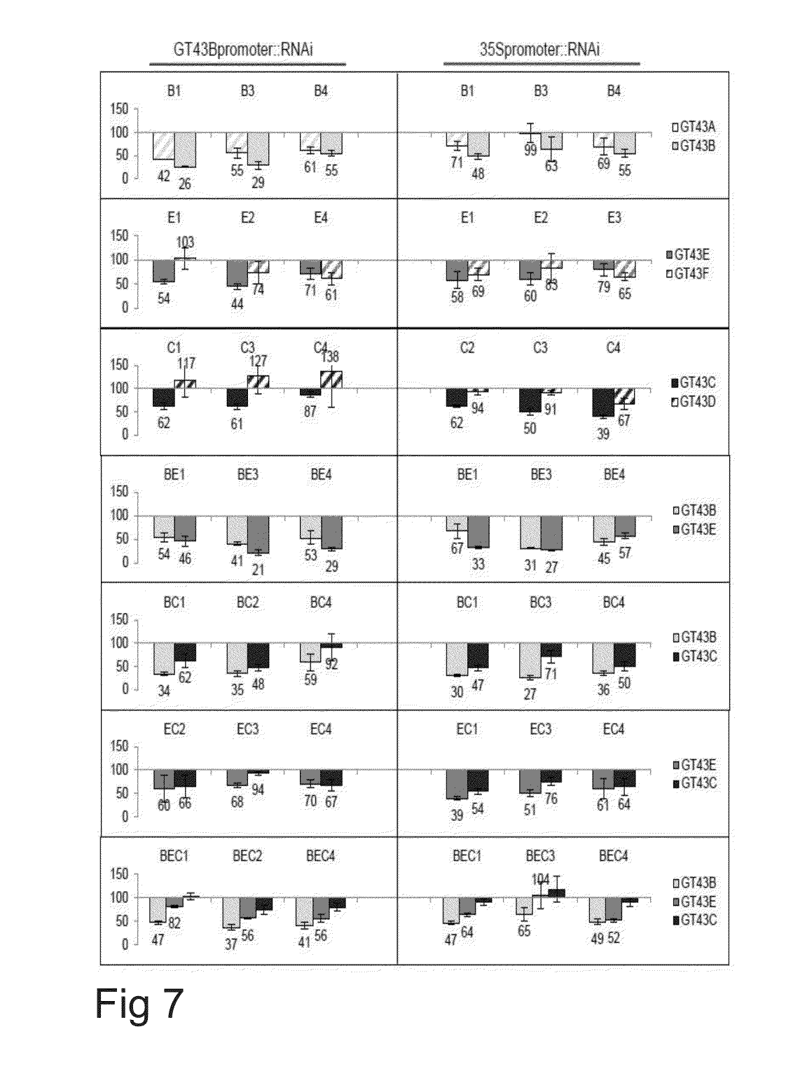

Details for different expression levels are shown in FIG. 7.

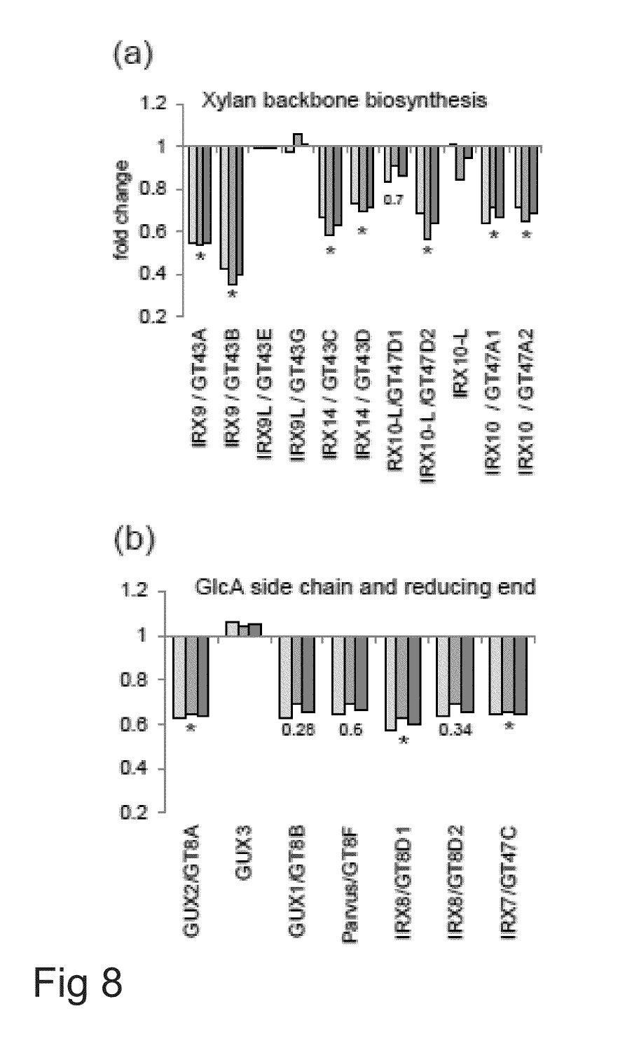

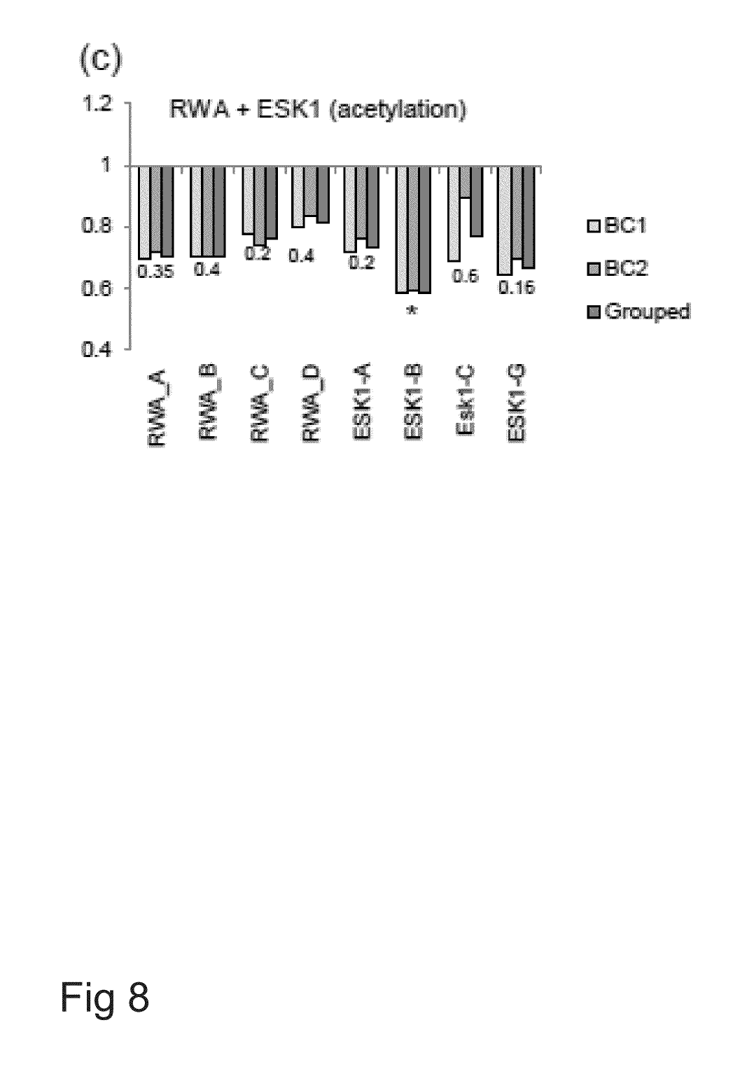

Aiming to understand the genetic regulatory networks in the WP::GT43BC RNAi lines and searching an explanation for the increased cambial cell division found in these transgenic lines, a study of the transcriptome in developing wood using RNA sequencing was performed. The targeted GT43 transcript reductions in GT43BC RNAi lines compared to wild type (FIG. 8) and the reduction observed in our qRT-PCR experiments (FIG. 7) were confirmed. All four gene members belonging to the IRX9 and IRX14 clades i.e. GT43A, B, C and D are successfully down-regulated in trees containing the GT43BC RNAi construct (FIG. 8). GT43E and G from clade IRX9-L were not affected by the RNA interference, which confirms the specificity of the RNAi construct. Looking at more xylan biosynthesis related genes, which are involved in xylan backbone, reducing end sequence formation and acetylation, other than the GT43 family, such genes overall had reduced transcript levels in the GT43BC RNAi lines compared to wild type (FIG. 8).

Furthermore, it was found that among the most down-regulated genes in the GT43BC RNAi lines were several genes related to secondary cell wall formation, including MYB transcription factors, cellulose and lignin biosynthetic genes and genes involved in sugar metabolism. In contrast, among the most upregulated genes we found factors involved in cambium maintenance and early xylem differentiation Thus, the reduction of GT43 expression leads to down-regulation of the cell wall biosynthetic machinery and to up-regulation of regulatory factors involved in cambium function.

Growth of the Transgenic Trees

Growth of transgenic lines was evaluated in the greenhouse experiments. Plant height, stem diameter, internode number and internode length were measured. In one of the constructs GT43BC, WP-driven, the growth was unexpectedly high resulting in a volume increase with 140% compared to a non-transformed hybrid aspen. This high growth increase is also expected to applicable in Eucalyptus, Acacia and Salix.

Furthermore, the construct GT43C showed significant difference between promoters for effects on plant height, stem diameter, and volume. The construct GT43C driven by the WP promoter increased with 120% compared to a non-transformed hybrid aspen, compared to only 85% when driven by the 35S promoter. In all these cases the growth was stimulated with WP-driven constructs compared to 35S-driven constructs.

To determine if the increased growth in the construct GT43BC with the WP promoter lines is caused by increased wood production, stem sections were examined under the microscope and the radius of pith, radial width of xylem and bark were measured. The transgenic lines had increased amounts of all three tissues, but the highest increase was observed in the thickness of xylem, more than 22% increase at the average.

Mechanical Properties of the Wood in Transgenic Lines

Trees with higher biomass yields are valuable for industrial applications. But if such trees are more brittle, and therefore easily damaged by biotic and abiotic stresses, they are not competitive.

Xylan-compromised poplars, i.e. poplar with reduced amounts of xylan are known to have reduced wood mechanical strength (Li et al. (2011) Tree Physiol. 31: 226-236). Therefore, the wood of transgenic lines was analysed for the modulus of flexural elasticity (MOE) and the bending strength (BS) in the three point bending test. Surprisingly, it was found that wood from the construct GT43BC with the WP promoter revealed higher MOE in both transgenic lines, and higher BS, when tested in two different tree lines. The improved mechanical properties were also supported by the bending strength in the three point bending test, which increased 11 to 18% compared to wild type, see example 8 for details.

A high elastic modulus means that the tested material is stiffer and requires more force to be elastically (non-permanently) deformed. Bending strength on the other hand represents the force applied at the materials moment of rupture. High bending strength in the transgenic tree thus implies high resilience of the wood.

Cell Wall Analyses in Transgenic Lines

To determine the nature of any cell wall alterations, the wood of transgenic lines and the WT was analysed by FT-IR. No significant changes were detected.

The neutral and acidic sugar composition in the wood of transgenic lines BC1, BC2, and BEC1 and the WT was determined by TMS analysis for changes in monosaccharide composition. No major changes were detected.

The hemicelluloses were further extracted from the wood cell walls and digested with xylanases to specifically analyse xylan. The released oligomers were then separated by polyacrylamide carbohydrate gel electrophoresis (PACE) and the signals were quantified by staining. The relative xylan chain length was determined by the ratio of signals from reducing end sequences to the signal from xylo-oligomers. The analysis revealed a decrease in xylan chain length by 10% in the construct GT43BC with the WP promoter line compared to WT.

Saccharification Analysis of Transgenic Hybrid Aspen Lines

The saccharification of wood of aspen with the construct GT43BC with the WP promoter was investigated by using two different approaches: (i) enzymatic hydrolysis without pretreatment, and (ii) pretreatment followed by enzymatic hydrolysis. Pretreatment was carried out using elevated temperature and addition of an acid catalyst [1% (w/w) sulfuric acid]. The monosaccharide yields were determined using high-performance anion-exchange chromatography. The transgenic aspen lines showed improved glucose production rates and improved glucose yields compared to the wild-type.

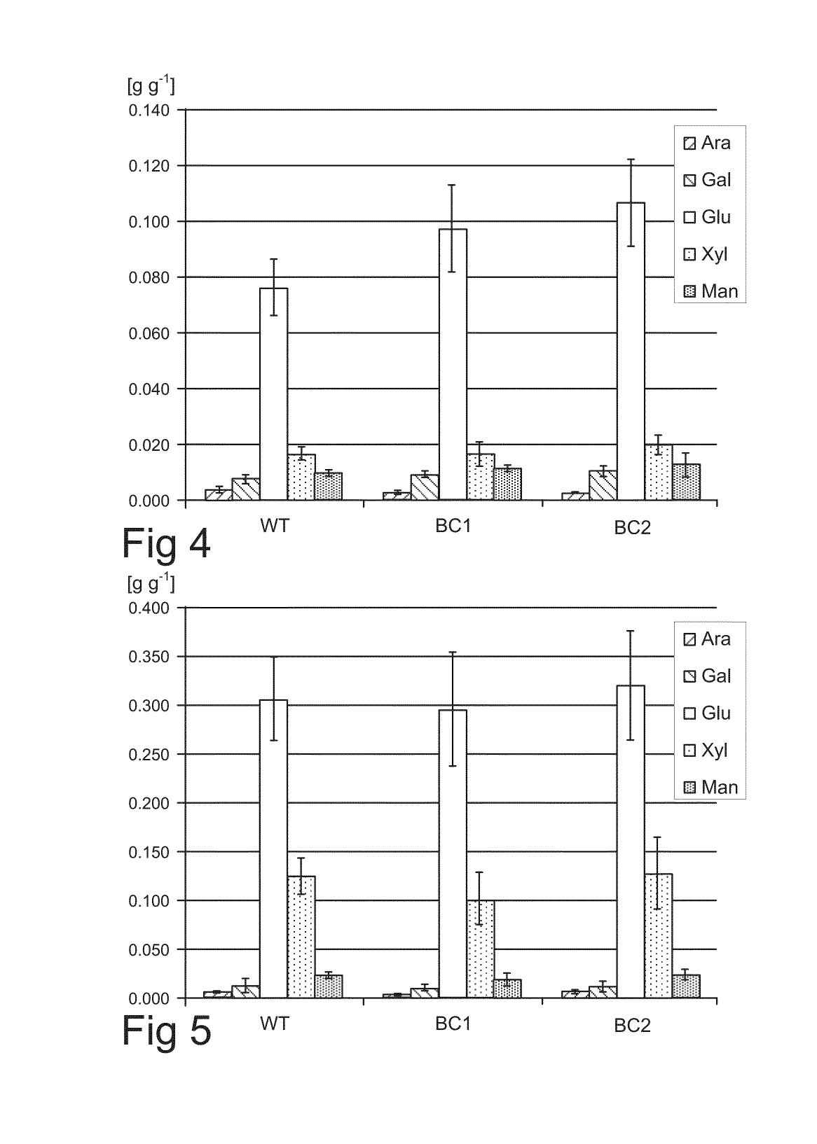

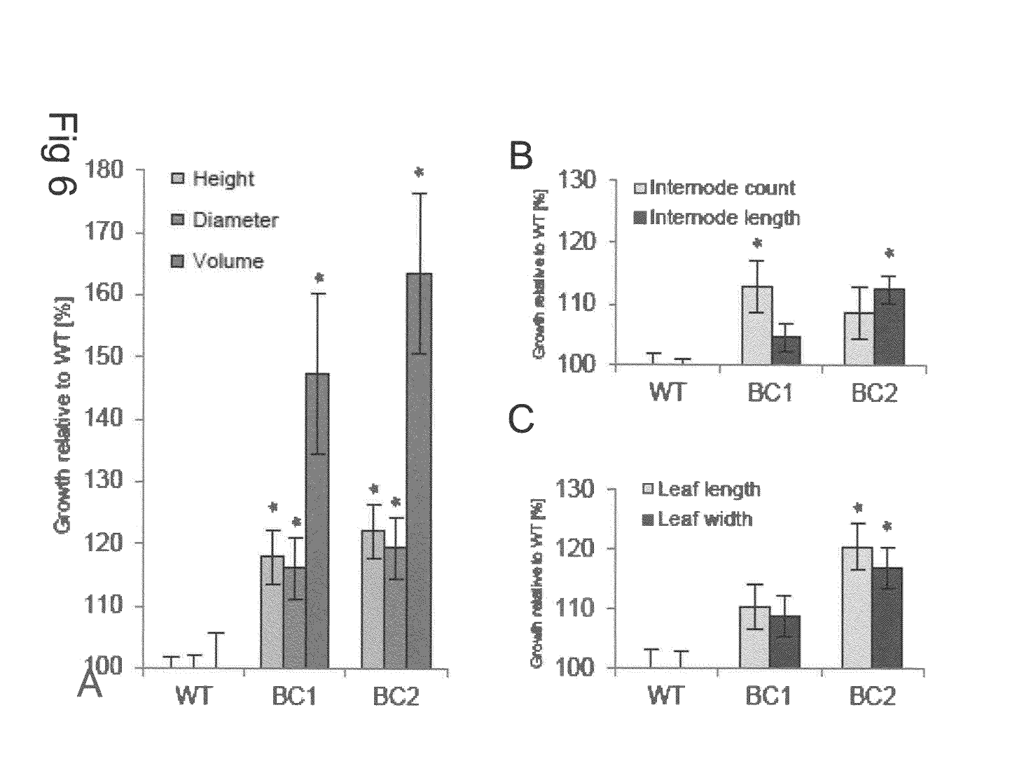

In FIG. 4 the results from saccharification experiments without pretreatment are shown. FIG. 4 shows the yields of monosaccharides after enzymatic hydrolysis (with standard deviation). The results show a significant increase (P≦5%, t-test) of the glucose yields of the transgenic aspens (BC1 and BC2). The increase in glucose yield corresponds to 27% for BC1 and 40% for BC2, which is unexpectedly high. The result from pretreatment followed by enzymatic hydrolysis is shown FIG. 5. No significant difference was detected (P≦5%, t-test) when comparing the transgenic aspens (BC1 and BC2) with the wild type.

### SUMMARY

These data show that a small decrease of xylan biosynthesis in woody plants leads to a number of positive effects in plants provided that the change is induced specifically in the cells forming secondary walls. This is achieved by using wood-specific promoter (WP) from the gene GT43B. The transgenic RNAi lines in which xylan synthase genes GT43B and GT43C were down-regulated to approximately 50-60% of WT but specifically in the secondary wall forming tissues under control of WP promoter, had only approx. 3-5% decrease in xylose content and an approximately 10% decrease in xylan chain length. Such lines grew unexpectedly better, had approx. 30-40% higher stem volume, approximately 30% increased module of elasticity (MOE), and approx.30-40% higher glucose yield in saccharification without pre-treatment.

The improved bioprocessing properties fermentation of such lignocellulose can be expected and be useful in fermentation.

Down-Regulation of Four GT43 Members (GT43A. B. C and D) Causes Increased Growth

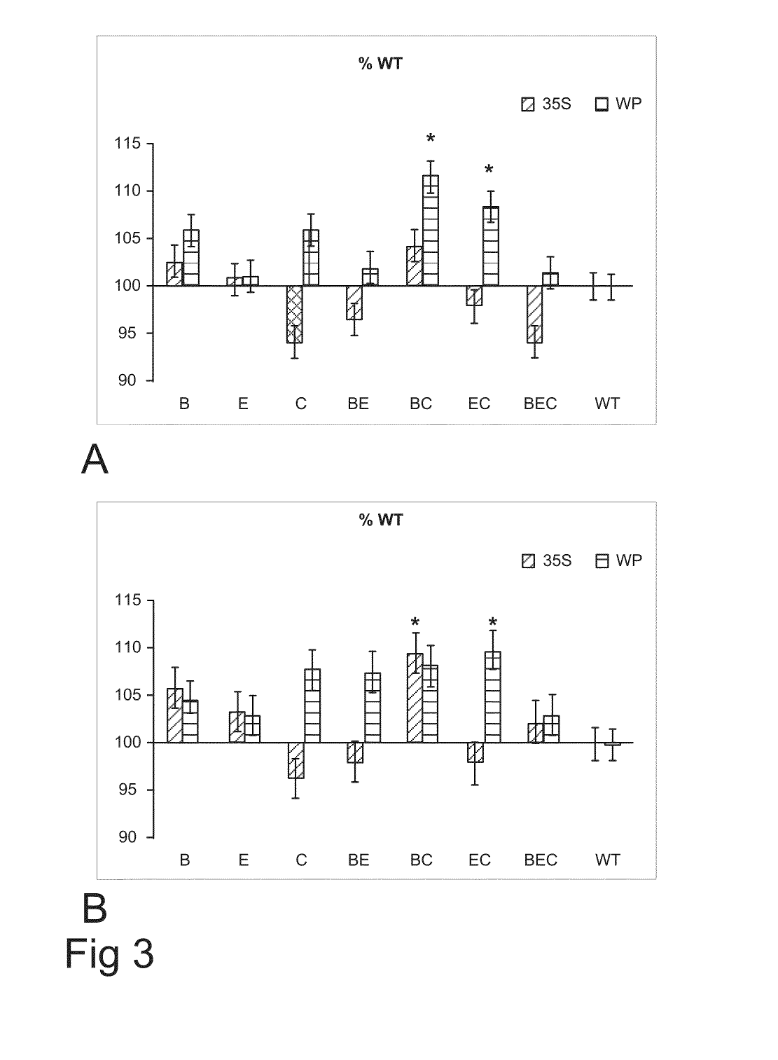

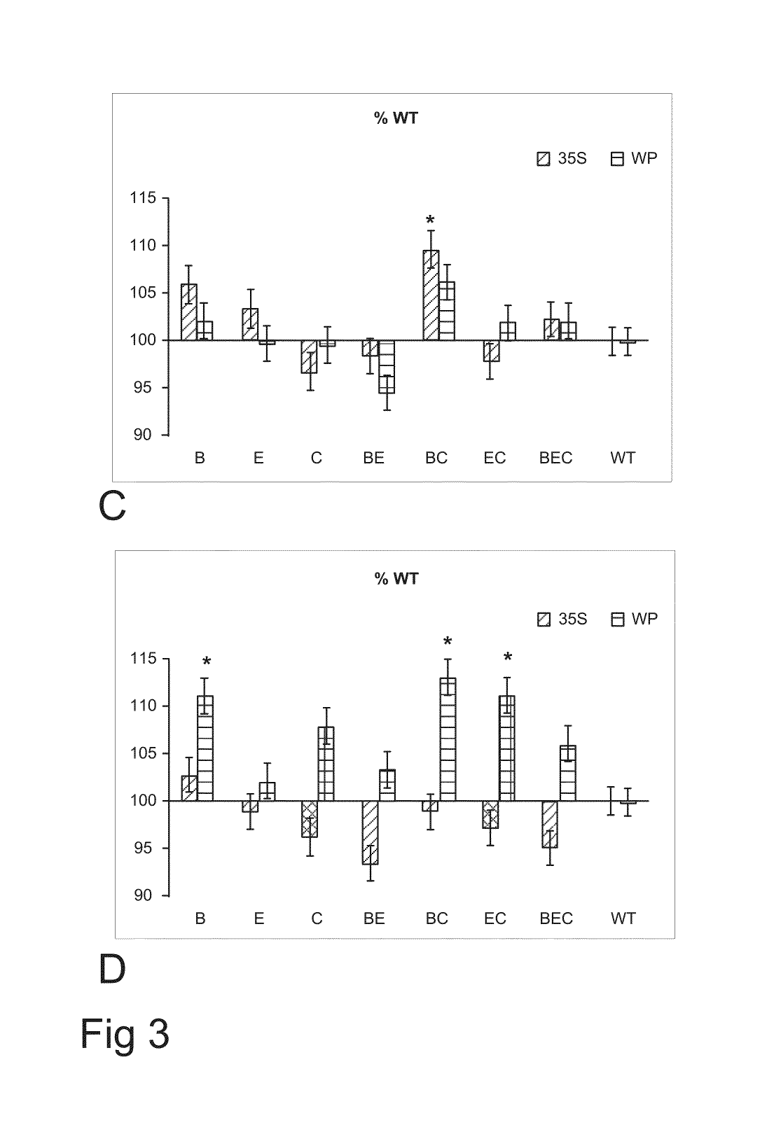

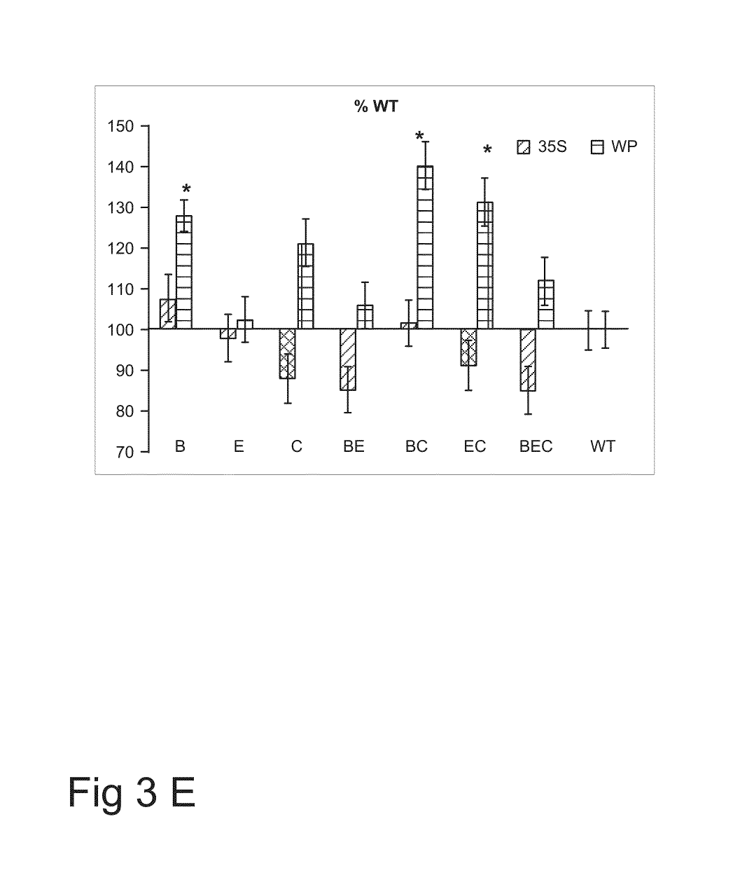

The pGT43B driven RNAi construct containing the GT43BC fragment targeting both IRX9 (GT43A and B) and IRX14 (GT43C and D) clades had most positive impact on growth compared to the other RNAi constructs (FIG. 3). Expression levels of the main gene in each clade, GT43B and GT43C were around 35% and 55%, respectively, of the wild type level in the two best lines GT43BC1 and BC2 (FIG. 7). Plant height and stem diameter of those lines were increased by 10-20% of the wild type level, resulting in a stem volume increase of 50-60% (FIG. 6). Also internode number, internode length and leaf size were increased by approximately 10%. Growth experiments in the greenhouse were repeated three times with randomised plant positioning for the two GT43BC lines 1 and 2 with similar results.

Stronger Wood and Lower Density

Trees with higher biomass yields are valuable for industrial applications. But if such trees are more brittle, and therefore easily damaged by biotic and abiotic stresses, they are not competitive. The mechanical properties of transgenic hybrid aspen wood with reduced GT43B and -C expression levels was thus tested. Based on published phenotypes of plants impaired in xylan biosynthesis, we expected impaired mechanical strength of the wood (Lee et al., 2011; Li et al., 2011) in a three point bending approach on carefully prepared specimens from mature wood. Surprisingly, we found that the tested wood of GT43BC RNAi lines had both higher elastic modulus and bending strength compared to wild type (FIG. 5). A high elastic modulus means that the tested material is stiffer and requires more force to be elastically (non-permanently) deformed. Bending strength on the other hand represents the force applied at the materials moment of rupture. High bending strength in the transgenics thus implies high resilience of the wood.

Plant Species

It is highly expected that when the expression of GT43 orthologous genes from other woody tree is reduced this will result in improved growth, mechanical properties and saccharification.

In accordance with the present invention, the transgenic tree is preferable a woody tree or a woody species. In a useful embodiment, the woody tree is a hardwood tree which may be selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood trees from the Salicaceae family, such as willow, poplar and aspen including variants thereof, are of particular interest, as these two groups include fast-growing species of tree or woody shrub which are grown specifically to provide timber and bio-fuel.

In further embodiments, the woody tree is a conifer which may be selected from the group consisting of cypress, Douglas fir, fir, sequoia, hemlock, cedar, juniper, larch, pine, redwood, spruce and yew.

In useful embodiments, the woody tree is a fruit bearing plant which may be selected from the group consisting of apple, plum, pear, orange, kiwi, lemon, cherry, grapevine and fig.

### EXAMPLES

### Example 1

### Cloning of Wood Specific Promoter from the GT43 Gene Family

In order to identify a good promoter from the GT43 gene family which is highly expressed in developing wood the transcript levels of all seven genes were quantified in vegetative aerial tissues by qRT-PCR. All seven promoters and WP (SEQ ID NOS: 23 to 30) were cloned as well as the 35S promoter was cloned into the GATEWAY cassette vector followed by the GUS gene for easy detection, resulting in eight different constructs. The sequences of the cloned promoters are shown as SEQ ID NOS: 23 to 30.

The nine pGT43::GUS fusion constructs were stably expressed in hybrid aspen and stems of two months old greenhouse grown trees were histochemically analyzed for GUS activity.

The staining for GT43A, GT43B and WP promoters is found in developing xylem and phloem fibers. In contrast, the activity of GT43C, -D and -E promoter is present in phloem parenchyma. In comparison, the activity of 35S promoter was detected in the bark as well as in developing wood and ray cells.

The construct WP::GUS turned out to have the most specific promoter and this promoter was selected for further studies.

To test the efficacy of the WP promoter, for genetically modifying wood, RNAi constructs using either WP- or 35S promoter were made in two different gene families to down-regulate specific family members.

### Example 2

### Transcription Level of all Seven GT43 Genes in Non-Transgenic Hybrid Aspen

The transcript levels in vegetative tissues of hybrid aspen (Populus tremula×tremuloides) for all seven GT43 genes were quantified in by qRT-PCR in vegetative aerial tissues. The results are summarized in Table I, above.

The mRNA transcript all seven genes were quantified in vegetative aerial tissues by qRT-PCR. Based on the non-normalized expression that allows comparing the expression levels of the different genes, it was found that genes GT43C and E were most highly expressed in developing wood, followed by genes GT43 B and A. Expression of GT43 F and G was not detected in this tissue.

Among the highly expressed genes, three genes were found to be specifically expressed in developing wood, xylem and tension wood: GT43A, GT43B, and GT43C. Expression of GT43A and B was more wood-specific than expression of GT43C.

### Example 3

### Transgenic Lines in Hybrid Aspen

Transgenic lines were generated in hybrid aspen designed to silence each of the GT43 clades using a clade-specific sequence of the most important gene for each clade: GT43B for clade I, GT43E from clade II and GT43C for clade III. These fragments were then combined to silence two clades, or all three clades at a time. All 7 types of inverted repeats were driven by either the CMV 35S or the WP promoters, resulting in 14 constructs that were transferred to the clone T89.

Approximately 20 independent lines per each construct were prescreened in vitro to select the three most affected lines that were multiplied along with the WT and planted in the greenhouse.

### Example 4

### Analysis of Expression of the GT43 Genes in Wood of Transgenic Lines

Expression levels of the different GT43 genes in the wood of transgenic lines that were selected were determined by qRT-PCR. The analysis showed that target gene expression was reduced in the wood of transgenic lines to the level between 35% and 75% of WT level at the average, and that there were no differences in the down-regulation level according to the promoter used in the construct.

### Example 5

### Growth of the Transgenic Lines

Growth of transgenic lines was evaluated in the greenhouse experiments. All transgenic lines, each represented by five trees, were grown in the greenhouse for approximately 3 months. The transgenic plants and the WT plants were uniformly distributed over the greenhouse growth rooms, and were rotated to eliminate effects of microenvironment in the rooms. Plant height, stem diameter, internode number and internode length were measured. Considering all the effects of the constructs, 35S promoter driven constructs did not affect growth except for increase in number of leaves and internode length in construct BC (Table IV). In contrast, WP-driven constructs increased height in constructs BC and EC, number of leaves in construct EC, stem diameter and stem volume in constructs B, BC, and EC. The biggest effect was observed in volume in construct BC reaching 140% of WT volume. Construct C showed significant difference between promoters for effects on plant height, stem diameter, and volume, and construct EC showed significant difference between promoters for stem diameter and volume. In all these cases the growth was stimulated with WP-driven constructs compared to 35S-driven constructs.

TABLE IV
Growth of GT43 RNAi lines.

| | | | | | | | | | | Diameter at 8 | | | | | |
|---|---|---|---|---|---|---|---|---|---|---|---|---|---|---|---|
| | Height | | | Leaf nr | | | Internode Length | | | weeks | | | Volume | | |
| | Mean % | P ≦ | Std | Mean % | P ≦ | Std | Mean % | P ≦ | Std | Mean % | P ≦ | Std | Mean % | P ≦ | Std |
| | WT | 5% | Error | WT | 5% | Error | WT | 5% | Error | WT | 5% | Error | WT | 5% | Error |
| 35S:RNAi GT43B | 103 | | 2 | 106 | | 2 | 106 | | 2 | 103 | | 2 | 108 | | 6 |
| 35S:RNAi GT43E | 101 | | 2 | 103 | | 2 | 103 | | 2 | 99 | | 2 | 98 | | 6 |
| 35S:RNAi GT43C | 94 | P | 2 | 96 | | 2 | 97 | | 2 | 96 | P | 2 | 88 | P | 6 |
| 35S:RNAi GT43BE | 97 | | 2 | 98 | | 2 | 98 | | 2 | 93 | | 2 | 86 | | 6 |
| 35S:RNAi GT43BC | 104 | | 2 | 110 | * | 2 | 110 | * | 2 | 99 | P | 2 | 102 | | 6 |
| 35S:RNAi GT43EC | 98 | | 2 | 98 | | 2 | 98 | | 2 | 97 | P | 2 | 91 | P | 6 |
| 35S:RNAi GT43BEC | 94 | | 2 | 102 | | 2 | 102 | | 2 | 95 | | 2 | 85 | | 6 |
| WP:RNAi GT43B | 106 | | 2 | 104 | | 2 | 102 | | 2 | 111 | * | 2 | 128 | * | 6 |
| WP:RNAi GT43E | 101 | | 2 | 103 | | 2 | 100 | | 2 | 102 | | 2 | 103 | | 6 |
| WP:RNAi GT43C | 106 | P | 2 | 108 | | 2 | 100 | | 2 | 108 | P | 2 | 121 | P | 6 |
| WP:RNAi GT43BE | 102 | | 2 | 108 | | 2 | 94 | | 2 | 103 | | 2 | 106 | | 6 |
| WP:RNAi GT43BC | 111 | * | 2 | 108 | | 2 | 106 | | 2 | 113 | P* | 2 | 140 | * | 6 |
| WP:RNAi GT43EC | 108 | * | 2 | 110 | * | 2 | 102 | | 2 | 111 | P* | 2 | 131 | P* | 6 |
| WP:RNAi GT43BEC | 101 | | 2 | 103 | | 2 | 102 | | 2 | 106 | | 2 | 112 | | 6 |

P - indicates significant difference according to the promoter used.
* indicates significant difference from the wild-type.
Significance is based on Tukey HSD tests.

The variability of growth in individual lines was also studied and most lines generated with the WP promoter outperformed the WT.

To ensure that the growth effects are stable over time, a separate greenhouse experiment was carried out with two selected lines WP:RNAi GT43 BC1 and BC2 for two months. Similar changes in growth were observed.

### Example 6

### Wood Formation in Transgenic Poplar

To determine if the increased stem diameters in WP:RNAi GT43BC lines is caused by increased wood production, stem sections were examined under the microscope and the radius of pith, radial width of xylem and bark were measured. The transgenic lines had increased amounts of all three tissues, but the highest increase was observed in the thickness of xylem, +22% at the average (Table V). Radial widths were measured in stem transverse sections under the microscope. P values correspond to the post-ANOVA contrast.

TABLE V
Stem diameter in WP: RNAi GT43BC lines

Thickness increase in % of WT

| | Xylem | Bark | Pith |
|---|---|---|---|
| WP: RNAi GT43BC line | +17% | +5% | +16% |
| BC1 | | | |
| WP: RNAi GT43BC line | +27% | +15% | +15% |
| BC2 | | | |
| | p = 0.003 | p = 0.026 | p = 0.003 |

### Example 7

### Fibre Width

To determine if the increased xylem width is caused by larger xylem fibres, the wood of transgenic lines and WT was macerated and the fibres were measured under the microscope. There was a small increase in fibre width in one of the lines, 7% in line BC2, but it could not have accounted for a 27% increase in xylem radial width in this line (compare Tables V and VI). No change in fibre width was observed in the line BC1, which had larger xylem, +17%. We conclude that the transgenic lines produce more xylem cells, which is the main cause for the increased stem diameters. No xylem abnormalities were detected by light microscopy in the transgenic lines.

TABLE VI
Fibre width

| | Fibre width [μm] | Change |
|---|---|---|
| T89 - WT | 20.7 | |
| WP: RNAi GT43BC line | 20.7 | ±0 |
| BC1 | | |
| WP: RNAi GT43BC line | 22.1 | +7% (p = 0.02) |
| BC2 | | |

### Example 8

### Mechanical Properties of Wood from Transgenic Lines

Xylan-compromised poplars are known to have reduced wood mechanical strength). Therefore, the wood of transgenic lines was analysed for the modulus of flextural elasticity (MOE) and the failure strength according to standard CSN EN 384, title “Structural timber—Determination of characteristic values of mechanical properties and density” (www.en-standard.eu/csn-en-384-structural-timber-determination-of-charactenstic-values-of-mechanical-properties-and-density/), or bending strength in the three point bending test (Esteves, B. M, Domingos, I. J. & Pereira, H. M. 2008. Heat treatment of pine wood. BioResources 3(1), 142-154). This revealed higher MOE in both transgenic lines (Table VII), and higher bending strength in both lines taken together (Table VIII). Percentages indicate significant difference in % from the WT by a t-test (P≦5%). Probability values correspond to the post-ANOVA contrast.

TABLE VII
Modulus of elasticity (MOE)

| | MOE (N/mm²) | % of WT | P-value |
|---|---|---|---|
| T89 - WT | 3607 | 100 | |
| WP: RNAi GT43BC line BC1 | 4664 | 130 | P ≦ 0.023 |
| WP: RNAi GT43BC line BC2 | 4887 | 135 | |

TABLE VIII
Bending strength (BS)

| | BS (N/mm²) | % of WT | P-value |
|---|---|---|---|
| T89 - WT | 57 | 100 | |
| WP: RNAi GT43BC line BC1 | 64 | 111 | P ≦ 0.095 |
| WP: RNAi GT43BC line BC2 | 68 | 118 | |

### Example 9

### Cell Wall Analyses in Transgenic Lines

To determine the nature of any cell wall alterations, the wood of transgenic lines and the WT was analysed by FT-IR. No significant change was detected between WP:GT43BC RNAi lines and the WT (data not shown).

The neutral and acidic sugar composition in the wood of transgenic lines BC1, BC2, and BEC1 and the WT was determined by TMS analysis. No major changes were detected. A small difference in xylose and Me-Glc A was noted, indicative of max. 5% decrease in xylan content, which was accompanied by a decrease in Ara and an increase in Glc.

### Example 10

### Xylan Analysis

The hemicelluloses were further extracted from the wood cell walls from the two tree lines from WP:GT43BC RNAi construct and digested with xylanases to specifically analyse xylan. The released oligomers were then separated by polyacrylamide carbohydrate gel electrophoresis (PACE) and the signals were quantified by staining. The relative xylan chain length was determined by the ratio of signals from reducing end sequences to the signal from xylo-oligomers (Table IX). The analysis revealed a small decrease in xylan chain length, by 13% in the more affected line WP:GT43BC2 RNAi, compared to WT. P value corresponds to the contrast between the transgenic lines and the WT.

TABLE IX
Relative xylan chain length. Means of three independent
experiments and SE, each with 3-6 biological replicates

| | Relative xylan | | |
|---|---|---|---|
| | chain length | Change | P value |
| T89 - WT | 213 | | |
| WP: RNAi GT43BC line BC1 | 201 | −6% | P ≦ 0.001 |
| WP: RNAi GT43BC line BC2 | 184 | −13% | |

### Example 11

### Saccharification Analysis of Transgenic Lines

The saccharification of wood of aspen was investigated by using two different approaches: (i) enzymatic hydrolysis without pre-treatment (FIG. 4), and (ii) pre-treatment followed by enzymatic hydrolysis (FIG. 5). Pretreatment was carried out using an elevated temperature and an acid catalyst [1% (w/w) sulfuric acid]. After pretreatment, the liquid fraction, referred to as the pre-treatment liquid, was separated from the solid residue. The solid residue was then degraded using an enzyme preparation. The monosaccharide yields (the yields of arabinose, galactose, glucose, xylose and mannose) were determined using high-performance anion-exchange chromatography

The results without acid pre-treatment (FIG. 4) show a significant increase (P≦5%, t-test) of the glucose yields of the transgenic aspens (BC1 and BC2). The increase in glucose yield corresponds to 27% for BC1 and 40% for BC2. With acid pre-treatment (FIG. 5), no significant difference was detected (P≦5%, t-test) when comparing the transgenic aspens (BC1 and BC2) with the wild type.

1. A method for producing a transgenic tree having reduced xylan content compared to a wild-type tree of the same species, said method comprising reducing expression of one or more genes from the glycosyltransferase 43 (GT43) family in the said transgenic tree, wherein said transgenic tree have increased growth properties and improved mechanical properties, and/or saccharification properties compared to a wild-type tree of the same species.

2. The method according to claim 1, comprising the steps of:
(a) introducing an expression vector into a tree cell, said expression vector comprising:
(i) at least one promoter;
(ii) a first nucleotide sequence comprising one or more genes from the GT43 family operably linked downstream of said promoter; and
(iii) a second nucleotide sequence which is complementary to the first nucleotide sequence operably linked downstream of said first nucleotide sequence;
wherein the said linked nucleotide sequences are transcribed into RNA in the cell, thereby producing a hairpin RNA which is processed in the cell to an interfering RNA molecule capable of reducing expression of one or more genes from the GT43 family; and
(b) culturing said tree cells in step (a) to a transgenic tree.

3. The method according to claim 2, wherein the said promoter is a wood-specific promoter which is up-regulated during secondary wall biosynthesis.

4. The method according to claim 3, wherein the said wood-specific promoter is selected from the group consisting of GT43 promoters; secondary wall CesA promoters; RWA promoters, especially promoters homologous to Arabidopsis RWA1, RWA3 or RWA4 promoters; promoters homologous to Arabidopsis IRX10 promoter; GUX1 or GUX 2 promoters; AtFRA8, AtlRX8/or AtPARVUS promoters; AtXyn1 promoter; AtTBL29 promoter.

5. The method according to claim 4, wherein the said wood-specific promoter is a GT43B promoter comprising the nucleotide sequence shown as SEQ ID NO: 25 or a GT43B-derived WP promoter comprising the nucleotide sequence shown as SEQ ID NO: 23.

6. The method according to claim 1, wherein in developing wood of a said transgenic tree, the level of mRNA transcribed from the said one or more genes from the GT43 family is between 25% and 85% of the corresponding levels of mRNA transcribed in wild-type tree of the same species.

7. The method according to anyone to claim 1 wherein the said one or more genes from the GT43 family are selected from
(a) the group of Populus trichocarpa genes consisting of:
GT43A (SEQ ID NO: 1),
GT43B (SEQ ID NO: 2),
GT43C (SEQ ID NO: 3),
GT43D (SEQ ID NO: 4),
GT43E (SEQ ID NO: 5),
GT43F (SEQ ID NO: 6),
GT43G (SEQ ID NO: 7);
(b) genes which are orthologous to the Populus trichocarpa genes in (a) and which have at least 80% sequence identity with the genes in (a).

8. The method according to claim 7, wherein the said orthologous genes in (b) are selected from a species selected from Eucalyptus, Acacia, or Salix.

9. The method according to claim 1 wherein the said one or more genes from the GT43 family are selected from the group consisting of:
GT43B (SEQ ID NO: 2),
GT43C (SEQ ID NO: 3), and
GT43E (SEQ ID NO: 5).

10. The method according to claim 9 wherein the said one or more genes from the GT43 family are selected from the group consisting of:
GT43B (SEQ ID NO: 2), and
GT43C (SEQ ID NO: 3).

11. The method according to claim 10, comprising reducing expression of both GT43B and GT43C or reducing expression of the three GT43 genes GT43B, GT43C and GT43E.

12. The method according to claim 1 for producing a transgenic tree having increased growth.

13. The method according to claim 12 wherein the increased growth is characterized by one or more features selected from increased height, increased stem diameter, increased stem volume, increased internode length, and increased number of leaves.

14. The method according to claim 1 for producing a transgenic tree having improved mechanical properties.

15. The method according to claim 14 wherein the said improved mechanical properties comprise increased modulus of elasticity and/or increased bending strength.

16. The method according to claim 1 for producing a transgenic tree, from which the yield of monosaccharides from hydrolyzed wood is increased compared to wood from a wild-type tree of the same species.

17. The method according to claim 16 wherein the said monosaccharides are selected from the group consisting of arabinose, galactose, glucose, xylose and mannose.

18. A transgenic tree obtainable by the method according to claim 1.

19. The transgenic tree according to claim 18 which is of a genus selected from Populus, Acacia, Salix or Eucalyptus.

20. An expression vector comprising:
(i) a wood-specific promoter which is active in developing wood;
(ii) at least one gene, optionally selected from the glycosyltransferase 43 (GT43) gene family.

21. An expression vector according to claim 20 comprising:
i. at least one promoter;
ii. a first nucleotide sequence comprising one or more genes from the GT43 family; and
iii. a second nucleotide sequence which is complementary to the first nucleotide sequence;
wherein the said nucleotide sequences are capable of being transcribed in a tree cell into a hairpin RNA structure which is processed in the cell to an interfering RNA molecule capable of reducing expression of a GT43 gene.

22. The expression vector according to claim 21 wherein the promoter is a GT43B promoter.

23. The expression vector according to one claim 20, wherein the expression vector further comprises one or more regulatory elements selected from the group consisting of transcriptor factor binding sites, splice sites and termination sites.

24. The expression vector according to claim 20, wherein the said first nucleotide sequence comprises two or three genes from the GT43 family.

25. The expression vector according to claim 24 wherein the said two genes are GT43B and GT43C or the said three genes are GT43B, GT43C and GT43E.

26. Use of the expression vector according to claim 20 for increased expression or for down-regulation of a gene, preferably a gene selected from the glycosyltransferase 43 (GT43) gene family.

27. A transgenic tree comprising the expression vector according to claim 20.

28. Wood obtainable from the transgenic tree according to claim 18.

29. A method for producing monosaccharides from wood comprising
(a) providing wood according to claim 28;
(b) degrading the wood; and
(c) obtaining free monosaccharides.


SEQUENCE LISTING (44 sequences)

SEQ ID NO: 1 - DNA, 1080 bp, Populus trichocarpa
atggggtctttggaaagatcaaagaagaaagttcaattatggaagaaagctatagttcattttggtttatgttttgttatgggcttttttacaggtttcgctccagggggcaaggcttcaattttttctagtcatgttgttgcatcaaataaatcgcaacctgttgaaatgttgcaccagcaggtagcaagtacaccacatgctagtaatgttaacagaagtttgatagctgaaagtcctgtgccaactccattaagctccaaggaatctgaacctgcaaaattcttggaaaaagaagaagaaccaaaacccaagttactaccaagaagactggcaattatcgttacgccaattagcactgaagatccgtaccaaggggtgtttttgaggaggttagctaatacaatcaggctggttccgccaccattattgtggattgttgtggaagggcaatcagattcagatgaagtatcggagatacttagaaaaacaggcatcatgtataggcatttggtcatcaaggagaattttacagatcctgaagcagagctggatcatcaaaggaatgttgctttaaggcacattgagcaacacaggctgagtggaattgttcattttgccgggctttccaatgtttatgatcttggcttctttgacgagcttaggcaaattgaggtgtttgggacatggccagtggctctactttcagcaaacaaaaacaaagtgacaattgaaggacccgtatgtgattcctcgcaggtcattgggtggcatctgaagaaaatgaacaatgaaacggataaaaggcctccaatccatatttcaagttttggattcaacagttcgatcctttgggatcctgagagatggggtcgcccttcatcagtccaacaaacctcacagaattcaatcaaatttgtcaaacaagcagctcttgaagatgaaacagagttgaagggaatcccaccagaggactgctcaaaaatcatgctttggcgtcttaatcttcccgtttcaaaatcaccaagctaccatctttcaaccaccggatccacggatgctagcaggagaaaaatatga

SEQ ID NO: 2 - DNA, 1074 bp, Populus trichocarpa
atgggctctgtggaaagatcaaagaggagagttcagttatggaagaaagctatagttcattttggtttgtgttttgttatggggttttttacaggtttcgctccagcaggcaaggcttcaatttttactagtcatgttgctgcatcaaataaatcgcaatccttgccgcaacctgttgaaatgacgttgcaccagcaggcagcaagtacaccacatgctagtaatgtcaacagaagtttgatagctgaaactgcagtaccagctccaccaagctctaaggaatctgaacatgcaacattcttggggaaagaagaaacagaatccaagttagcgccaagacgactggcaataatcgttacgccaacaagcactaaagatccgtatcaaggggtttttttgaggaggttagctaatacgatcaggttggttcctccaccattattgtggattgttgtggaagggcaatcagattcagatgaagtatcggaggtccttaggaaaacaggtattatgtataggcatttggtgttcaaggagaattttacagatcccgaagcagagttggatcatcaaaggaatgttgcattaaggcacattgaaaaacacaggctgagtgggattgttcattttgccgggctttccaatgtttacgatctaggcttctttgacgagattagacaaattgaggtgtttggaacatggccaatggcattactttcagcaaacgagaagaaagtgataattgaaggacctgtatgtgattcctcgcaggttatcggctggcatctaaggaaaatgaacaacgaaacggataaaaggcctccaattcatatttcaagttttggattcaacagctcaatcctctgggatcctgagagatggggtcgcccttcttcagtccagcaaacctcacagaactcaatcaaattcgtcaaacaagtagctcttgaagacgagacaaagttaaagggaatcccaccagaggactgttcaaaaatcatgctttggcgtcttaatcttcctacttcaaaatcaccaagttatcaggaaaatcaagaagacaaaatcgtgtag

SEQ ID NO: 3 - DNA, 1533 bp, Populus trichocarpa
atgaaattctccttgctacagcagagctacaacaaccgccgaagcggcagtttcagaggatcatctgcgccgttagattcatcaccggacaacacaatcaaatcaccagccgctattttctggctctttttacatggcatttgttgtctcatcagtctcgtcctcggctttcgcttctctcgtctcgttttcttcttcctcttctctacctccacgaccacaactctctatgtaacgacaccgtttcaccctctcagcaaaacatccgacatttccaatcccttaaccaatagtgctaatgatcttccggtgattaacaaaacggtgagttcgagagttgttgtcgggcggcacgggatccggatccggccatggccgcacccgaacccgagtgaagtaataaaagcgcatcagataatagagagagttcaaagagagcagagtaatcaattcggtgtgaagagtcctaggagtcttatcgtagtcacgcctacttacgtgcggactttccagactttacatatgactggtgttatgcattctctgatgttgctcccctacgacgtcgtctggatcgtggtggaggccggtggggtcaccaatgaaacagcattgattattgccaaatccggcgttaagactcttcatattggatttaatcagaaaatgccgaattcttgggaaggaaggcacagattggagactaaaatgcggcttcgcgctttgagagttgtgagagaggagaagatggatggaattgtgatgtttgcggatgatagtaatatgcatagtatggagctgtttgatgagatacaaaatgtgaagtggtttggtgcggtttcagttgggattcttgttcattcaggtggtgctgatgagacgttattaactgctgctgctgctatggttgacaaggaggcggaagaaaatttgcctaatccagtagtgcctgttcagggtccggcttgtaatgcttcaaacaaattggttggttggcatacgtttaattcgttaccgtatgagggaaagagtgcggtatatattgatgatagggctacggtgttgcctaggaagctggagtgggctgggtttatgttgaattcgaggcttctttggaaggaagctgaagataaaccagaatgggttaaggatatggatttggttgatgagaatatagagaatcctttagctctgctgaaggatccttccatggtggagccacttgggagctgtgggcgacaagttttgctctggtggctccgtgttgaggctcgtgctgatagcaaattccctccagggtggataattgacccacctttggaaattactgtgccatcgaagcgaacaccatggcctgatgcccctcctgaactgccttccaatgaaaagatatcagttaaccaagaacagactgctaagcgttcttcaaagactcgatcacccagatcaaaacgcagttcaagaagtaagagaaagcatgaagtagtgttggcggagacacaggtttctgcaaggcattctgaacaaaattga

SEQ ID NO: 4 - DNA, 1512 bp, Populus trichocarpa
atgaaactctccatgctacagcagagctacatgaaccgccgaagcgcaagttttagaggatcatcagcacccttagattcatccacagacaacacaatcaaatcaccagccgcgatcttttggctccttctacatggcttctgctgtctcatcagtcttgtcctcggctttcgcttctctcgtctcgttttcttcttcctcttctccacctccaccactacaactctctacatagcgacaccgctcccccatctcaccaaaacgaataataacatcaatgatctcccccttgaaatcccggttatcaacaaaacgctgagttcgagttccagagttgtggtcgggcgtcacgggatccggatccggccatggccgcacccgaacccgagtgaagtaatgaaggcgcatcagataatagagactgttcagagagagcaaaggactcaattcggtgtgaagagtcctaggacccttattgtagtcacgcccacttatgtacggactttccaaactttacatttgactggcgttatgcattctctgatgttggtcccctacgacgtcgtctggatcgtggtggaggccggtggtgccaccaatgaaactgcttccattatcgccaaatcgagtatcaagacttttcatattggatttactcagaaaatgccgaattcttgggaaggaaggcacaaattggagactaaaatgcgacttcgagctttgagagttgtgagagaggagatgatggatggaatagtgatgtttgcggatgatagtaacatgcatagtatggaactatttgatgagatacaaaatgtgaaatggtttggtgcggtttcggttgggattctggctcattctggtggtggtggtgagtcgtcttcagctgtggctgagaaggatgtgaaaccgaatttgtctaatccagcaatgcctgttcagggaccagcttgtaatgcttctaacaaattggttggttggcatacgtttaattcattgccgtatgaggggaagagtgccgtttatatcgatgatagagctacagtgttgcctagaaagcttgagtgggctgggtttgtgttgaattcaaggttgcttttgaaggaagctcaggataagcctgaatgggttaaggatcttgatttggttgatgagaatatagagagtcctttagctttgctgaaggatccttctatggtagagccacttgggagctgtgggcgacaagttttgctgtggtggcttcgtgttgaagctcgtgctgatagcaaattccctccaggatggataattgacccaccgttggaaattactgtgccatcaaaacgaacaccatggccagatgctcctcctgaactcccttccaacaaaaagttaacaattaaccaagagcagactattaagcgttctccaaagactcgatcaccccgatcaaaacgcagaagtaagagaaagcatgaagcaaagttggtggagacacaggtttctacaaggcattctgaacaaaattga

SEQ ID NO: 5 - DNA, 1188 bp, Populus trichocarpa
atggcatcaatccgtcgaacactctcacaggtttaccaagaccgcagctaccaaaacggcgtcgcttcagcacaagctcacaagctcttcagcactaacaacaacagcggcaagtattcttccttgacctccacctccgccgtcgcagcagcttccgtctatctccgccgcaaaggatttcgcagatccttttataggtgcacgatattcttcatcctagggcttttacttggtatcttcccgtttggtcaagtggataacgatatcaataagcatgatttctccttcgaaatgaagccgccacatgtcaatgtccaattggacaccaaagataatttcgctctcgctgccgtgtccctcggcgtcgagaaaacgacaccgcaattggatcgattttcaaggtttgattatgtagagcgaaagcaagtgatagtgataacaccgacatataatcgagcattacaagcctacttcttgaatagattaggtcaggtgttgaggttagtgcagccgccgttgttgtggattgtggtggagatgacgtcagcatcggcggagacagcggagatattgaggaaaaccggggtgatgtataggcatttggtttgtgttaataagaataacacgaatgtgaaggacagaggagtgcatcagaggaatgcgggattggagcatatagaaaggcatagacttgatgggattgtgtattttgctgatgatgataatgtatattcgcttcaattgtttgagagcttgagaaatatcagtcattttggtacttggcctgttgcaatgcttgcacaaagcaaaaacaaagcaattgtggaaggtccggtatgcaatgccagtcaagtaattggatggcacacaaatgagaaaagtaaaagactccgcaggtttcatgttgatatgtctggatttgctttcaacagcaccatcttgtgggacccaaagagatggaaccgccccttttcaaatccaattcgacagttagatacagtgaaggagggttttcaagagaccacattcattgagcaagtggtagaagatgaaagtcaaatggaaagtgtaccacccagctgttcaaggatactgaactggcatcttcatttagatgctcatggtcttgtctatcccagaggctggctgctccagaagaacctagaagttgttcagcccatcaagtga

SEQ ID NO: 6 - DNA, 666 bp, Populus trichocarpa
atggcatctataaggagaacattgtctcctgtgcctagagctggaaccttattaaacggggaagcctgtcaagtggcttctcctttgtccaagtcctcttattcccagagctacccctcttctggtggattgctaccatcaattttcggtccgtcagattctcaagcctttgtttatggtgttttttcaccaagatcttcaagacctttggagaggtcaaaacccgaggggcaagtttggaagagggccctttcccatttcttcgtttgttttgtgattgggttcagaacatggacagttgccaagttaacagtaaacagaaacaaagatttcgtggagggctccatttgtaatggaactcaagctattggatggcatgtgaatgaatcaagtagaagatttcagagattcgatgctggcatgtcagggtttgccttcaatagtatcatcatttgggatccaaaacgatggcatcgtccaactcctgaacctatcaggcagcttgaaatagtcaaggatggcttccaggtttgcacatttattgagcaagttgtggaacatgaaagccaaatggaaggcttactggaggactgctcaggagtcatggcctggcatctgcagcttcaatttcacattctagggagtatatcacctccgattggttag

SEQ ID NO: 7 - DNA, 1329 bp, Populus trichocarpa
atggcatctataaggagaacattgtctcctgtgcctagagctggaaccttattaaacggggaagcctgtcaagtggcttctcctttgtccaagtcctcatcgtcgtattctcagagctacccaacttctggtggatttctatcttcaatttttggtttatcagacgttcaagcttttgcttatggtgttttttcaccaagatcttcgagaccattggagaggtcaaaatccaaggggcaagtttggaagagggcccttttccatttcttggtttcttttgtgattggggtttttattggactaaccccatttgtctcgatgaatttatcaacgaatcccatgtcaaagcaccaagcgttttcatttgaggtggtatctactgttgggaattttgataagcatgaagatatgacaagaaatgcaacaacaatagcagaaaggggaggacttgagaacagtaccgcactggagccacaagtgaaggaagaggagtcaggggatgggaattctaatggtacctccattagcctatcgctctctgaagatgtgaatttggtgtctcggaaactcttgatcattgtgaccccaactcatgctcgaccacttcaagcctattatctgagtcgtttggcgcacacattaaaactggttcaacctcctctattatggatagttgtggaaatgacattgcaatctgatcatacggctgatatcctgaggaggactggggttatgtataggcatcttgtttgcaacaaaaatttaactgatataaaagacagaagtgttcaccaaaggaatgtggcattgtctcacattgaaatccaccaccttgatggaatagtccattttgcagatgattataacacttactctgcagatctttttgagcaaatgaggcagatcaggcggtttgggacatggacagttgccaagttaacaggaaacaaaaacaaagattttgtagagggccccatttgtaatggaactcaagttattggatggcatgtgaatgactcaaggaggagatttcggagattccatgctgacatgtcggggtttgccttcaacagtaccatcatttgggatccaaaacgatggcaccgccctacgcccgaacctattaggcagcttgacacagtaagggatggcttccaggttagctcatttattgagcaagttgtggaagatgaaagccaaatggagggcttattggaggactgctcaagagtcatggtctggcttctgcagcttcaatcatctaattccttgtatcctcctaagtggttccttgatgttaatctggatgtcatcacccaggctgcatga

SEQ ID NO: 8 - protein, 359 aa, Populus trichocarpa
MGSLERSKKKVQLWKKAIVHFGLCFVMGFFTGFAPGGKASIFSSHVVASNKSQPVEMLHQQVASTPHASNVNRSLIAESPVPTPLSSKESEPAKFLEKEEEPKPKLLPRRLAIIVTPISTEDPYQGVFLRRLANTIRLVPPPLLWIVVEGQSDSDEVSEILRKTGIMYRHLVIKENFTDPEAELDHQRNVALRHIEQHRLSGIVHFAGLSNVYDLGFFDELRQIEVFGTWPVALLSANKNKVTIEGPVCDSSQVIGWHLKKMNNETDKRPPIHISSFGFNSSILWDPERWGRPSSVQQTSQNSIKFVKQAALEDETELKGIPPEDCSKIMLWRLNLPVSKSPSYHLSTTGSTDASRRKI

SEQ ID NO: 9 - protein, 357 aa, Populus trichocarpa
MGSVERSKRRVQLWKKAIVHFGLCFVMGFFTGFAPAGKASIFTSHVAASNKSQSLPQPVEMTLHQQAASTPHASNVNRSLIAETAVPAPPSSKESEHATFLGKEETESKLAPRRLAIIVTPTSTKDPYQGVFLRRLANTIRLVPPPLLWIVVEGQSDSDEVSEVLRKTGIMYRHLVFKENFTDPEAELDHQRNVALRHIEKHRLSGIVHFAGLSNVYDLGFFDEIRQIEVFGTWPMALLSANEKKVIIEGPVCDSSQVIGWHLRKMNNETDKRPPIHISSFGFNSSILWDPERWGRPSSVQQTSQNSIKFVKQVALEDETKLKGIPPEDCSKIMLWRLNLPTSKSPSYQENQEDKIV

SEQ ID NO: 10 - protein, 510 aa, Populus trichocarpa
MKFSLLQQSYNNRRSGSFRGSSAPLDSSPDNTIKSPAAIFWLFLHGICCLISLVLGFRFSRLVFFFLFSTSTTTTLYVTTPFHPLSKTSDISNPLTNSANDLPVINKTVSSRVVVGRHGIRIRPWPHPNPSEVIKAHQIIERVQREQSNQFGVKSPRSLIVVTPTYVRTFQTLHMTGVMHSLMLLPYDVVWIVVEAGGVTNETALIIAKSGVKTLHIGFNQKMPNSWEGRHRLETKMRLRALRVVREEKMDGIVMFADDSNMHSMELFDEIQNVKWFGAVSVGILVHSGGADETLLTAAAAMVDKEAEENLPNPVVPVQGPACNASNKLVGWHTFNSLPYEGKSAVYIDDRATVLPRKLEWAGFMLNSRLLWKEAEDKPEWVKDMDLVDENIENPLALLKDPSMVEPLGSCGRQVLLWWLRVEARADSKFPPGWIIDPPLEITVPSKRTPWPDAPPELPSNEKISVNQEQTAKRSSKTRSPRSKRSSRSKRKHEVVLAETQVSARHSEQN

SEQ ID NO: 11 - protein, 503 aa, Populus trichocarpa
MKLSMLQQSYMNRRSASFRGSSAPLDSSTDNTIKSPAAIFWLLLHGFCCLISLVLGFRFSRLVFFFLFSTSTTTTLYIATPLPHLTKTNNNINDLPLEIPVINKTLSSSSRVVVGRHGIRIRPWPHPNPSEVMKAHQIIETVQREQRTQFGVKSPRTLIVVTPTYVRTFQTLHLTGVMHSLMLVPYDVVWIVVEAGGATNETASIIAKSSIKTFHIGFTQKMPNSWEGRHKLETKMRLRALRVVREEMMDGIVMFADDSNMHSMELFDEIQNVKWFGAVSVGILAHSGGGGESSSAVAEKDVKPNLSNPAMPVQGPACNASNKLVGWHTFNSLPYEGKSAVYIDDRATVLPRKLEWAGFVLNSRLLLKEAQDKPEWVKDLDLVDENIESPLALLKDPSMVEPLGSCGRQVLLWWLRVEARADSKFPPGWIIDPPLEITVPSKRTPWPDAPPELPSNKKLTINQEQTIKRSPKTRSPRSKRRSKRKHEAKLVETQVSTRHSEQN

SEQ ID NO: 12 - protein, 395 aa, Populus trichocarpa
MASIRRTLSQVYQDRSYQNGVASAQAHKLFSTNNNSGKYSSLTSTSAVAAASVYLRRKGFRRSFYRCTIFFILGLLLGIFPFGQVDNDINKHDFSFEMKPPHVNVQLDTKDNFALAAVSLGVEKTTPQLDRFSRFDYVERKQVIVITPTYNRALQAYFLNRLGQVLRLVQPPLLWIVVEMTSASAETAEILRKTGVMYRHLVCVNKNNTNVKDRGVHQRNAGLEHIERHRLDGIVYFADDDNVYSLQLFESLRNISHFGTWPVAMLAQSKNKAIVEGPVCNASQVIGWHTNEKSKRLRRFHVDMSGFAFNSTILWDPKRWNRPFSNPIRQLDTVKEGFQETTFIEQVVEDESQMESVPPSCSRILNWHLHLDAHGLVYPRGWLLQKNLEVVQPIK

SEQ ID NO: 13 - protein, 221 aa, Populus trichocarpa
MASIRRTLSPVPRAGTLLNGEACQVASPLSKSSYSQSYPSSGGLLPSIFGPSDSQAFVYGVFSPRSSRPLERSKPEGQVWKRALSHFFVCFVIGFRTWTVAKLTVNRNKDFVEGSICNGTQAIGWHVNESSRRFQRFDAGMSGFAFNSIIIWDPKRWHRPTPEPIRQLEIVKDGFQVCTFIEQVVEHESQMEGLLEDCSGVMAWHLQLQFHILGSISPPIG

SEQ ID NO: 14 - protein, 442 aa, Populus trichocarpa
MASIRRTLSPVPRAGTLLNGEACQVASPLSKSSSSYSQSYPTSGGFLSSIFGLSDVQAFAYGVFSPRSSRPLERSKSKGQVWKRALFHFLVSFVIGVFIGLTPFVSMNLSTNPMSKHQAFSFEVVSTVGNFDKHEDMTRNATTIAERGGLENSTALEPQVKEEESGDGNSNGTSISLSLSEDVNLVSRKLLIIVTPTHARPLQAYYLSRLAHTLKLVQPPLLWIVVEMTLQSDHTADILRRTGVMYRHLVCNKNLTDIKDRSVHQRNVALSHIEIHHLDGIVHFADDYNTYSADLFEQMRQIRRFGTWTVAKLTGNKNKDFVEGPICNGTQVIGWHVNDSRRRFRRFHADMSGFAFNSTIIWDPKRWHRPTPEPIRQLDTVRDGFQVSSFIEQVVEDESQMEGLLEDCSRVMVWLLQLQSSNSLYPPKWFLDVNLDVITQAA

SEQ ID NO: 15 - DNA, 1056 bp, Arabidopsis thaliana
atgggatctctagagagatcaaagaagaaagctcaagtatggaagaaagctgtgatccatttctctctatgttttgtaatgggtttcttcactggcttcgctcctgctggtaaggcctcatttttctctaatttcgaaacgacttcttatacttctacaaaatctccgattcctccccaaccttttgaaaacgctacatacacacaacattccctcctcaatagaaccttgatcaattcccaaagccaagctccagctcccgcagagtcgcgagaagctgaaggagaaacaagatctctctccgagaaagaagatgagaatcaagtaaaagtgacaccaagaggactagtgattgttgtaactccaataataactaaagatcgctacaaaaacgttcttcttaggagaatggccaataccttgaggctagttccacctcccttattgtggatagtcgtggaaaaacactcggatggcgaagaaaaatcttcgtctacaatgttaagaaagaccggtatcatgtatagacgtatagtcttcaaggaagatttcacaagcttagaatcagagcttgatcatcaaaggaatctagctttgaggcacattgaacatcacaaattaagcggaatagttcattttgcgggactaaacaacatatatgatcttgattttttcgtcaagatcagagatatcgaggtatttggtacttggccaatggcattgttatcggctaataggaaacgagtggtagtagaagggcctgtttgtgaatcttcacaagtattggggtggcatttgagaaaaattaataacgagacagagacaaagcctccgattcatatatcaagcttcgctttcaatagttccatactttgggaccctgagagatggggtcgtccttcttctgttgaaggcaccaaacaggattcgataaaatatgtgaagcaagtagttttggaggacgatacaaagttgaagggacttccggcacaagattgttccaagattatgctttggcgtctcaaatttcccacaagaacacgtttaagcacctga

SEQ ID NO: 16 - DNA, 1185 bp, Arabidopsis thaliana
atggcgtcaatccggcgaactctctcgccgatgtatcacgatcgttctcacgagaatggaggttcacacaaaggtttcaccattggtggtagtagcagcaaacacaattcttcgcaatttctttcgtatctcaccaagctcctcggagtaacgtctgatcctaaaagctcgcgtagaggtccatggaggcgaccgttttaccagtttctcgtcttctttcttcttggatttgtattaggtttgacacctttcggaaaaatggaagatgtaaacggttccgatcgattctccttcgagatcaaacagccctacgttgaagaacggttggaaaataggaaacgggaagaagcagctgtagatgcggtgagtttcgtggcggagactgaaaatgggaagaaggaggtcaattttgtgccgaagaagcttctaattgtggtcacaccaacgtataatagagcgatgcaggcgtattacttgaacagggttgctcaaacgctgagactagttgagtcgcctgtgctgtggatagtggtggagggtaacgtggcgtcgtttgaaacctcagagattctgaggaagacgggagtgatgtacaggcacttggtttgcaagaggaacatgacgagtatcaaggatagaggagtgcatcagagaaacactgcattggaacatattgagttgcataagcttgatggaattgtctactttgctgatgatgacaatatctactcgcttgagctctttcaaagcttgagacaaatcagccgatttgggacttggcctgttgctatgctcgcacaaagcaaaaacaaggccatccttgaaggtccagtatgcaatggaagtcaagtaataggatggcacactaatgaaaagagtaaaagactaaggaggttccatgttgatatgtcgggatttgcttttaacagcactatactctgggaccctaaaaggtggcgacgtcccttttcacatccaacccgccaactagacacagtgaaggagggtttccaggagacatcatttatagagcaggtggtggctgatgaaagcgagatggaaggtgttccaccagcttgctcgagtatactgaactggcatcttcacttggatgccctggatgtcccttatccacaagggtgggcgatccaaaagaatctgcaggctctcataactatgaaatag

SEQ ID NO: 17 - DNA, 1578 bp, Arabidopsis thaliana
atgaagctctctgctttacatcagagttacttaaatcgccggagtaatagcttcagatctccgacgtctcttgattcttccgttgatggctccgggaagtctttaatcgctgtgttttggcttatcctgcactgtctttgttgcttgattagtctagttctcggctttcgattctccagattagtcttcttcttcctcttctctacttcttcaaccaatctctactctcttccgtttcgtcctgacttacctgtgaaacacctcgatgttcacacaatcggccgtactctcgatcccggagctaacggaacgacggtggtggcgacggcgacgaaaagctctcgtgttgttgttggaagacacgggatccggatccgtccttggccgcatccgaatcccgttgaggtaatgaaagctcatcagatcattgggagagttcaaaaagagcaaaagatgatctttgggatgaaaagtagtaagatggttatagctgtgacaccgacttatgtgaggacttttcaagctttgcatttgactggtgtgatgcattctttgatgcttgttccttatgatctggtttggatcgttgttgaagctggtggtgctactaatgagaccggtttgattattgcaaaatcaggacttaggaccattcatgttgggattgatcagagaatgcctaatacttgggaagatcgtagtaaattagaagtctttatgagacttcaagctttgagagttgtgagggaagagaagcttgatgggattgtgatgtttgcggatgatagtaatatgcatagtatggagttgtttgatgagattcagaatgtgaagtggtttggtactgtttctgttgggatattggctcattcaggaaatgcggaagagatggttttgtcgatggaaaagaggaaagagatggagaaagaggaagaagaggagagctcttcgttacctgtacagggtcctgcttgtaactcaactgatcagttgattggttggcatattttcaatacattgccatatgcggggaagagtgcagtttatatagatgatgttgctgcggttttgcctcaaaaactagagtggtctgggtttgtgttgaactcgagattgctttgggaggaagctgagaataagccagagtgggttaaggactttgggtcgttgaatgagaatgaaggtgtggagagtcctttgtctctgttgaaggatccttcaatggtggagcctcttggaagctgtggaagacaagttctgctatggtggcttcgagtagaagcacgcgctgatagcaaattccctcccggatggataattgatcctccgttagagatcacagttgcggctaaacgcactccatggccagatgttcctcctgagccaccaactaaaaagaaagatcaaatgccattatcccaaggcaacaccgtcgtggtaataccaaagcagcaacaacatccaacgaaaatccgaaaaccgaaacgcaaaagtaagaaaagtaaacacgaacctagaccaaccgatacaacaacacaagtttattcatcttcgtctaagcatcaagaaagaaactga

SEQ ID NO: 18 - DNA, 1479 bp, Arabidopsis thaliana
atgaagctctctgtgtttcgattgagctattggaaccgtcgaggaagtagtttcagatcatcgccgtcgttggatccatcattcgatggcaaatctccgtcgtctgtgttttggttcgtgattcatggtctctgctgcttgatcagcttgattctagggttccgattcagccatttagtactcttcttccttttctcgacttccgtcaccaatctatacacaacgccatttctctttgccggaaacggcggtgtaagccagcttctccggctaaaacctctggaaacagcgactaacagcacggtgaagaagaactctcgagtggtggttggaagacacgggatccggatccgtccatggcctcacccgaatccgattgaggtattgagagctcatcagttgcttgtgagagtacagaaagagcagaaatcgatgtacggtgtgaggagccctaggactgtgattgtggtgacgccgacttatgtacggacttttcaggcgcttcatttgaccggagttatgcactcgcttatgcttgttccgtacgatttggtttggatcgttgtggaagctggtggaatcactaacgagactgcttcgtttatcgcaaaatcaggattaaagacgattcacttaggattcgatcagaaaatgcctaatacatgggaagatcgtcacaaattggagaccaaaatgagacttcacgccttgagagttgtgagagagaagaagttagatgggattgttatgtttgctgatgatagcaatatgcatagtatggagctttttgatgagattcaaactgtgaaatggtttggtgctctatctgttggtatacttgctcattctggtaatgcagatgaattatcatcgatcttgaagaatgaacaagggaagaacaaagagaaaccttcaatgccaatccaaggtcctagttgtaattcctctgagaaattagtgggttggcacattttcaacacacagccttatgccaagaagactgcagtgtatatcgatgagaaagcgcctgtgatgcctagtaagatggaatggtcagggtttgtgttgaattctagattgctctggaaggaatctttagatgataaaccagcatgggttaaagatctcagcttgttggatgatggttatgcggaaattgagagtcctttgtctttggtgaaggatccttccatggtggagccacttggaagctgtggccgtcgtgtcttgctttggtggcttcgagttgaagctcgagctgatagcaaattcccacctggctggatcataaagtcacctttagaaatcacagtgccatcaaagcggacaccctggccagactcttcctcagagctcccagcggcggcgatcaaagaggcaaaaagcaactctaagccaagagtgtcgaagagcaagagctataaggagaaacaagaacctaaagctttcgatggtgtcaaagtgtcagcaactagctga

SEQ ID NO: 19 - protein, 351 aa, Arabidopsis thaliana
MGSLERSKKKAQVWKKAVIHFSLCFVMGFFTGFAPAGKASFFSNFETTSYTSTKSPIPPQPFENATYTQHSLLNRTLINSQSQAPAPAESREAEGETRSLSEKEDENQVKVTPRGLVIVVTPIITKDRYKNVLLRRMANTLRLVPPPLLWIVVEKHSDGEEKSSSTMLRKTGIMYRRIVFKEDFTSLESELDHQRNLALRHIEHHKLSGIVHFAGLNNIYDLDFFVKIRDIEVFGTWPMALLSANRKRVVVEGPVCESSQVLGWHLRKINNETETKPPIHISSFAFNSSILWDPERWGRPSSVEGTKQDSIKYVKQVVLEDDTKLKGLPAQDCSKIMLWRLKFPTRTRLST

SEQ ID NO: 20 - protein, 394 aa, Arabidopsis thaliana
MASIRRTLSPMYHDRSHENGGSHKGFTIGGSSSKHNSSQFLSYLTKLLGVTSDPKSSRRGPWRRPFYQFLVFFLLGFVLGLTPFGKMEDVNGSDRFSFEIKQPYVEERLENRKREEAAVDAVSFVAETENGKKEVNFVPKKLLIVVTPTYNRAMQAYYLNRVAQTLRLVESPVLWIVVEGNVASFETSEILRKTGVMYRHLVCKRNMTSIKDRGVHQRNTALEHIELHKLDGIVYFADDDNIYSLELFQSLRQISRFGTWPVAMLAQSKNKAILEGPVCNGSQVIGWHTNEKSKRLRRFHVDMSGFAFNSTILWDPKRWRRPFSHPTRQLDTVKEGFQETSFIEQVVADESEMEGVPPACSSILNWHLHLDALDVPYPQGWAIQKNLQALITMK

SEQ ID NO: 21 - protein, 525 aa, Arabidopsis thaliana
MKLSALHQSYLNRRSNSFRSPTSLDSSVDGSGKSLIAVFWLILHCLCCLISLVLGFRFSRLVFFFLFSTSSTNLYSLPFRPDLPVKHLDVHTIGRTLDPGANGTTVVATATKSSRVVVGRHGIRIRPWPHPNPVEVMKAHQIIGRVQKEQKMIFGMKSSKMVIAVTPTYVRTFQALHLTGVMHSLMLVPYDLVWIVVEAGGATNETGLIIAKSGLRTIHVGIDQRMPNTWEDRSKLEVFMRLQALRVVREEKLDGIVMFADDSNMHSMELFDEIQNVKWFGTVSVGILAHSGNAEEMVLSMEKRKEMEKEEEEESSSLPVQGPACNSTDQLIGWHIFNTLPYAGKSAVYIDDVAAVLPQKLEWSGFVLNSRLLWEEAENKPEWVKDFGSLNENEGVESPLSLLKDPSMVEPLGSCGRQVLLWWLRVEARADSKFPPGWIIDPPLEITVAAKRTPWPDVPPEPPTKKKDQMPLSQGNTVVVIPKQQQHPTKIRKPKRKSKKSKHEPRPTDTTTQVYSSSSKHQERN

SEQ ID NO: 22 - protein, 492 aa, Arabidopsis thaliana
MKLSVFRLSYWNRRGSSFRSSPSLDPSFDGKSPSSVFWFVIHGLCCLISLILGFRFSHLVLFFLFSTSVTNLYTTPFLFAGNGGVSQLLRLKPLETATNSTVKKNSRVVVGRHGIRIRPWPHPNPIEVLRAHQLLVRVQKEQKSMYGVRSPRTVIVVTPTYVRTFQALHLTGVMHSLMLVPYDLVWIVVEAGGITNETASFIAKSGLKTIHLGFDQKMPNTWEDRHKLETKMRLHALRVVREKKLDGIVMFADDSNMHSMELFDEIQTVKWFGALSVGILAHSGNADELSSILKNEQGKNKEKPSMPIQGPSCNSSEKLVGWHIFNTQPYAKKTAVYIDEKAPVMPSKMEWSGFVLNSRLLWKESLDDKPAWVKDLSLLDDGYAEIESPLSLVKDPSMVEPLGSCGRRVLLWWLRVEARADSKFPPGWIIKSPLEITVPSKRTPWPDSSSELPAAAIKEAKSNSKPRVSKSKSYKEKQEPKAFDGVKVSATS

SEQ ID NO: 23 - DNA, 1492 bp, Populus trichocarpa
cacctcacctcacctcacctcttctctattcttttttctctttccaagttatcaatgaataaaatatatgatttaatcatttattaagtggttcagtgataagagtttgaaattaaaggattttttttttatggtcttaatttcgagccatgcggttactaatatgatgaccagtggagacttacatgatcgttaacttcagggcctgtaagatcagtcgaattacacgcaagctggctcagacacccacgttaatatatatatatatatatatatatgaccaacttcatgatcttactcgataaattcattttaaaatatgaaatcatcatcacaagtatccatttaaattacttaatgaataatataattaacttaattatttcatatacaccggtcactttctagtttctgtaactatatattttgaattttcaattcttttcactttcttttgatggaaaggtttttaattcataataattggattggacttgtctattttctttagagatttatcaattgtgttttttttttaacatttattaaattaaatagactagaataatactatacataccttatctatgatgtatttagtattgcgttccgttgaacaaattttgaaatttatttttgtttaaaattaattttttttatgttttcggatcattttaatgtgctgatatcaaaaataattttgaaaaaataaaaaaaatattattttaatacatttctgaactaaaaacactttaaaaaataactattaccacaattccaaacaccctagatcaacatattaagacagcaaggtcaaattaaagggtaataaaaggagagttttttttttttttggttggtcggttttggacctaatcagacaatcaaaaaaaaaaatcatggatcaaaatgtacgtaattcaatcatggtgtgtaatagattttttatatgatcatgtgtcaccagctaggtctttttccaaaattctaacaagaggttggtatatgtaataaaattatctttaaacagacacaactcacaccctctataattaatcttaacaacaagttaatataaaacaagtttttttttttcctaaatatattttgctgatgggtagcatcccaccaactatctggtgcagatagcaactcaagtccttcccaacgaacatatttttccatgatttctttcacttcaagtgacaactaaacgaagcaaagcctcgtcaaattaaaaaaaaagggttccgaataatcataatgtggcttttgagtttgccagtacctgtcatcgaccatttagcctcaacctccacaaaaaaacacacaacttcgctataaaacgactccttgtatattaattgttgtcttccacatctctcaatctctctcgaagaatccctttctttttccccaaaactcagtggtcagtatctgagccaacagcttggattttgatcacgtttttgaggggtttagcttgatgggc

SEQ ID NO: 24 - DNA, 1472 bp, Populus trichocarpa
atgcttttctgcctgtttgattttatcttctcttctcttcaagttttgagaacagtttacaatttatctttacagtttatgcgtgttttggaattttacaagccgagcttgttatgttttttggttgtttgagacttgttcgtgaatttaagttctctggtatccagttatctttacctgcatcatctcagtcatcaatgtttgtataattgttggtcttgccagttttctggctttgctctggcccttgctattgatttagatgagaaaataacgttcccgatgctgtcacaaaaactaaagcttcaactggaaacgatgctttcccatccttgtacaagatgatgatcccctgtttcaactttcgaaggaatggacatgcctttggagcctgaaggaatttgctttggtgttcttactatcctgctgtccgaaattagatgggtggaagcttttatgcgccccactcaagctgttgaaaccataacatccctcgttacatcacggaagctgcccatctttgacgtgaagatgaagaagctatgatagcagttttgactggaaatgttgtagtgattaaatgtttgttattgagtggagaagaataattagacacggacaacgtatccatcatcatcagttggaacaagtaacaaattgctattaatagtataatccatttcatgttgttcgagtcagctttctctctcaaaggtctcttttttcctggtattttgatcctccctccctccctccctccctccaagaaattacaaagggaaatcctaggctgcaaatcccctataagaagacttcagaaccaaatcaaggattataagaacacatgctttgaattaccatgtatggaaatataaatacctaaagccagagcattcaagccttttgaaaccatttttactcactgggaaatgctgctcaacttcagctcataagtagacaatacccgggacagacaacatctgataccctctatactcggtttacataattaatctcaacaacaggtgaatataaaataaagtgtttttccaggtcttatttagttgatgggttgcatcccaccaactatttggtgcagacagcaactcaagcccttcccactagcctctacacacatttttttacgttttctttcacttcaagtgataactaaacgaagcaatgccttgtcaaatatgttccaaaaataaaataaaaaaaacatgccttttgagtttgccagtaccttcctttcatggacaagttagcttcaaccttgacaaaaaaaaaaaaaaaaaaacaattatatatccttcaatataaagcgactcccttttacattaattttagtcttctacatttatcactctctcccccaaagaatccccttctttttttgccaaaactcagtgatcaatatctagctaaccaaacccagaaacttagattttgaagggtttagctaggtgggg

SEQ ID NO: 25 - DNA, 1516 bp, Populus trichocarpa
cacctcacctcacctcacctcttctctattcttttttctctttccaagttatcaatgaataaaatatatgatttaatcatttattaagtggttcagtgataagagtttgaaattaaaggattttttttttatggtcttaatttcgagccatgcggttactaatatgatgaccagtggagacttacatgatcgttaacttcagggcctgtaagatcagtcgaattacacgcaagctggctcagacacccacgttaatatatatatatatatatatatatatatatatatatatatatatatatgaccaacttcatgatcttactcgataaattcattttaaaatatgaaatcatcatcacaagtatccatttaaattacttaatgaataatataattaacttaattatttcatatacaccggtcactttctagtttctgtaactatatattttgaattttcaattcttttcactttcttttgatggaaaggtttttaattcataataattggattggacttgtctattttctttagagatttatcaattgtgttttttttttaacatttattaaattaaatagactagaataatactatacataccttatctatgatgtatttagtattgcgttccgttgaacaaattttgaaatttatttttgtttaaaattaattttttttatgttttcggatcattttaatgtgctgatatcaaaaataattttgaaaaaataaaaaaaatattattttaatacatttctgaactaaaaacactttaaaaaataactattaccacaattccaaacaccctagatcaacatattaagacagcaaggtcaaattaaagggtaataaaaggagagttttttttttttttggttggtcggttttggacctaatcagacaatcaaaaaaaaaaatcatggatcaaaatgtacgtaattcaatcatggtgtgtaatagattttttatatgatcatgtgtcaccagctaggtctttttccaaaattctaacaagaggttggtatatgtaataaaattatctttaaacagacacaactcacaccctctataattaatcttaacaacaagttaatataaaacaagtttttttttttcctaaatatattttgctgatgggtagcatcccaccaactatctggtgcagatagcaactcaagtccttcccaacgaacatatttttccatgatttctttcacttcaagtgacaactaaacgaagcaaagcctcgtcaaattaaaaaaaaagggttccgaataatcataatgtggcttttgagtttgccagtacctgtcatcgaccatttagcctcaacctccacaaaaaaacacacaacttcgctataaaacgactccttgtatattaattgttgtcttccacatctctcaatctctctcgaagaatccctttctttttccccaaaactcagtggtcagtatctgagccaacagcttggattttgatcacgtttttgaggggtttagcttggtgggc

SEQ ID NO: 26 - DNA, 1561 bp, Populus trichocarpa
tccctagatttaggcggtgttgagataaaattatcattgacatcgattcaagctactaaaataatcgatttttgtgtcaaataatttcatttgacgaaagaaattcaacttaggtgtttatttttattttttaccttgagtacctaaaaaaacatatatgagcttgagaataattttgtttcggttttttagatttttttttttgggtttttaagtctaatgatggatttatcaagattcctataggtttgtctaggtgttttttttttgtcaaaaatgaattgaataatttttagggtaaaaaaatccaatcttgattttccagttaccacaacggtcaactgggctaagtcaggtatcacacttaatttttttcaaaaaagcattggtgtcgttagccccaattatatttaaaaaaaaaaaggcccatgtttaggcttgcctttgcaagcccgtgcgcaagcccttttaaatgggccatgcccggtgatatcttaactctttctgttttgttttggtaattttggttttttttttaaatcatgcttttttacatgtattttttctaaatgttttcttaatttatatttaaattaatatctcttatctctaattttttattttgtatttagtattttttataaaaaaattgtggttgttttttaagtattttgtttgtattttattttttaatagatttttctaattcatttataaatttttatgttttatataattaaactttatttttaaaataaaaaattatttattcttggataaaatttctaataagtgcaatattgcgagatattttttttctttgattttattcaatcaattttatatgtttttatttctaccatcatttaattaaataaaaaataattttaataatagagtcattaattacaatcgtgtaaatgaaccatgttgtgatatcaaatattttgatctaaattcgttttttgactttttcagatacatatttagtttatcgttaataatattttatttatttattaatatagatgattttaatttatttaattagatgtttacataaaattatttttagtgaaaataatcagttagtgctacaaattaagtaagtaaaaaataaaaaaagaaataagtaaatgtaaaacagataggagtagtatttactggatcccaaaaattaagccaaaaaataatcaattgtcctaaacgaaacaattaactaatccacctggcccctaatcatgaacgccccccctttcttctcatcagtgtccactcgctgtctttaggtatcaaaacagcagcagcgcaacaacaataataaaaaataataatagtaataataataaaaaatcaaaaccaaccgacaaattctttcttcaatttcaattcaaagaaatctctcttctacttcgtcttcttcctaagcacaccttaacaagcttcaaacaaactcaacaaatacctctcctttccttctcatctcacaaatcccccactttctatctctaaactaaactctaccactttagagagagaaagagagccacgaaaagtgaaa

SEQ ID NO: 27 - DNA, 1498 bp, Populus trichocarpa
gatcgatctgggtcaactcgagttaatttttcaaattcaaaacctgggtcatgagaccatgattactccatagaaaacaaattgatacaaattatgaatatcaatccccaattaattcaaagttaaatgatgaaattgaaaaaatcaaataaaaaaataactcgtctcaactcgaattaactttccaaacacatgacccgggttatgagatccggataacctcatagaaagcaaattgaaacaaattatgcaccttagttataaaaaaattcaatgttgaagaatgaaattaaaaaaaataattaaaaaaagacacaaaaaaatttcagagtcgacttaagttaacttgccaaactcataacccgggttatgagattgtgataactttaaagaaaataaatcaaaataaatcatgaaacctaattcttaaactaccaaaatgttgaaggatgaaattaaaaaaataacttattaaaaaaaggatagaaaatgactcgagttaaccacgttaaacctactaaacccgtgactcgggtcatgcggttagtataatctgatagaaaacaaaatgaaataaattacaaagaacaattctcaattaaccgaaagttgaaagatgaaattgtaaaaagatttaattaaaaaaaataaaaaaatataaattaatctaactaacaagtcaaattcgtttcgtgacaatataattatctcataaaaaaattaaaataatttataaaacttaaaatagagcaaactcaatatgaaaagaagaactaaattaaagaaaacaaatgaatagcaactcgtgaagtctcattcaaggtttgttataattgtatcaaagtctttgtcaaagaatatatataaatgaatatataaaataaaaacgtctggcttcttacaacgaactatgatggcagctaatgtacgatacggtccataaaatatgagagagcgtaatttcaactaaagctcgagtaaatgaatatataaaataaaaactttgccgcagctttttatttcaagggttaactaaattaatactaatttgtaataataacaagagctctgtgcttcaaaaatctattaatactaatttgcacaatatgtaattatttttagttgttaagtaattagtcacaaattaagaaaaatgaggaattaggtatttatttgctcaatccaaaaaataattaatgtcctaaatgaattaattaacaaatccacccagacatgagctcgtgcctctaccctcctccccccttcctctcatccctgtcttatgcccttacaaacagccacagcaggacagtgggaaaaaaataaatccctcctccttctttttaaacagtaaaaaacaaattaaaaattaaaaaaaaaaaaagacctcaacgaacttgaagcaagcacctctcctctccatctcctctctcatatctcacccaccttctctctacaaactaaactcttagagagagagagagagagaagagtgaaa

SEQ ID NO: 28 - DNA, 1551 bp, Populus trichocarpa
tgttgacagtgcgtgtagaggattttgggtgttagattggataaatataatggtgagttttgtgcaataacatagtgaagatttgttgaaaacatttgtcttgatgtgttttttaatttgatgtttgtgtttattagagattttaaaggacaagagagatcatttgtgaatagttgtgttcaccggaaatgtcgagagaagagggattgaatttgttataaacatgaagttggtgatattttgtaaatccaaatccatgtctaacaactattttttcacaaaaattgacaactatcatgtagctatgaccaacgtcatgtagtggcaattttaattttaatttttaaaaaacaagaactctttaatcattaattgaaaataacaacaaacttgtaaatattatgatatttttttaattttttttgcatcagaggatgttaagttttataaaattattaaagttaaggaaataaaatatattttttcaaaaaattaagataaagcaaaaaatcgatcaaattataaaattctctatgtaagtatcccgaaataaaaagaggttatttttgtttttagtgtcaaatattgtccttaacttgttttaagtaataaaatcttatttttttcttcttcaaatcaagtgttaattcccggcaagttatcaaaataaattatcaggtttaaaaagacaggattgacaaaacaattaggtgattataatagaaaatgcttcaaagattaaaagaaaaggaaagtgaaagaaatacagtgcaaatccataattaacactgagttaattttataataattaaaaagtaattatttgcatcttattaattaattcattaaagaaaaaagatggatacaaagggaaggaaaaaaaaaactatatgtaaatatgattattacttttagtgagtgttaaatttaatttttttctttcctagtataaattaagttatctttttatttaagataaatttaatagaaaaactaataaaatattattaattaattactgacacaccaaaaaatcttccatgaaatgtgatttgatccacagtattttcatgaactgaattagcttttgtttttttttcaagtaattgtaaagagtccatgtgtgcgactgagatatacgggaagagacgcgtagactttccaagtacaagcaaccacttgtcgttcgttaaatggaacaccaccgcgtaatttagcgctcaatcaacccatctctccagctgggccgtactccgcccccagctatccctgcaacgatctcctcttcccctctccgcgctagaaatattaaaaaaagggggcaccagatcttagccttccatctctgaaatccactaccatcgcctctaaaagcatccgaacaaaatcttgaaatctaaaatccattttcactctataatttttttaaaaaaactgaaaatcataaattccatttaacttcattttagggttttttcatgtggattagtctccatcgtccatcaaatcttgatttgataatgtaagctgctcgaaaccaatcaagtggca

SEQ ID NO: 29 - DNA, 1551 bp, Populus trichocarpa; identical to SEQ ID NO: 28

SEQ ID NO: 30 - DNA, 1455 bp, Populus trichocarpa
tcctgtttgtttttacgttccaaaatgcttttaaaaaaatttaaaaatttttaatttttttatttgtttcaaattaatatttttttagtgttttaaaattattttgatatgttgatataaaaaataaattttaaaaaataaaaaaaaatattattttaatatatttttaaataaaaaatattttaaaaaataactagatagagaaaaaggttatttttgaaatgttaaacaatttaactctacaacttggttataaaacctagattagcaagataggtccgaaaattgatgagccagaatacagactaatccgagttaaaaaatatatgttaaaaagaatgttaaaaacttgagttaacctaaaattgagtctgaagctaagttttataacaagtgtagacattataatcattacgctaaatttatattcccctcaggaggtacctattacgtaagtgtaattaataataactcactcattaattacttccagaaagatacaatgtcaattattaattactccaataattaccagtgcccaattgcattgaaaaaaaattcttaaagtttctagtgcctgaatcatcatgattggcagagacagagtatgtgatgtgaagtattcctatcggatgacatcactctccctctctctctctctcctgtgtctaactcagattcctattccattttcaaagtttaagtctttctctgttcactctttctaattcttgatccttaaaacatcaaatactttagagggtgtatatacttaactagtgaaacttttcaacaagacccaaagcagcgtatttgtcaaatcttgaaaaaaatgtggtctttagctcctaagatccaggacccatttaccattgtagggtaatttcaagacccatttcgtttcactgatacttccaagcattttgggcatcgatctccttcctttgagggtatgtacttacctagtatttttggttttacggtttgtttaggtgaagatttgtgttgtggagctcattatagatcactgacttgtgaaattgcattttggggtttggttttagctatttatgactttgtttgaggttgcaagagaaagttgctttgctctgcttaaaggggtagttatacatgaacttcggtccccataaactgtttgcaacataggagacaggttttaaattttggcaatccaagatttgttgcttggccatgtgtgtctagtcaagggtgtattttgggttccaagcagttgttaagatattgaagagagtgtatgaaagagaacatctttatcggctttgaatctttatatttatggtgattagttggtaggatattgagattttaagtggatttggatgctctttttgttgtagttgtaatactgatttgaaattgtaaggaggtggatctcgaggttagtgatattccggtggattagtttgggtggca

SEQ ID NO: 31 - DNA, 1131 bp, Eucalyptus grandis
atggggtctgtggaaagatcaaagaagaaggtccagttgtggaagaaagccattgtccacttctctttatgtttcgtcatgggattcttcaccggcttcgctcccacgggtaaggccaggacgttcgctggccaacccgtcatgtccaacaaaatagagatcttctctccacaacctggagaactatcggacccgccaccgaccacatcttcgcacgtcagtttcaatagaagcttgatttctgaggtgccggagcgtgtctcggggaaaccccgtgccgagcctgaagaggacgatctcttggagaacgaagataaccaattagaagaagctgaatttccagagggaaacaataagcttgtccctaggaggctcttgatcattgtcacacctaccacaaaccatccattgacaggtgtgctgctgagaaggttggccaatactataaagttggtccctcaaccattgctgtggatcatcgtcgaaaagcggtcggactccagggaagtgtcggagatgatgaggaagacaggaatcatgtaccggcacttggtcttccgcgagaacttcacggacccggatgcagagatggaccaccagcggaaccttgctctccggcacatcgagcaccacaaactcagcggtattgttcacttcgctgggctttcaaacgtctacgatctggacttcttccaggagctcagagagattgaggtgtttgggacgtggcccatggcggtgctatcggcgaaccggaagaaggtgaacatagaagggccagtgtgcgactcgtcccaagtgctgggatggcacctgaggagattgagcaacgaaaccacggccacgagcgaatcgagcgagagacctcccattcacatctccagcttcgcattcaacagctccatcctttgggaccctgaacgatggggtcgcccttcttccggtcaacccaactctcaggactccatgaagttcgtgaaagaagtggtgcacgaggacgagaccaaagtgaagggacttccaggagagaagtgctccaagatcatgctctggcatcttcgctacctccttcctcgtcctcatcatcctccctcgacagctctccatcagacgctgacttctcaacgataa

SEQ ID NO: 32 - DNA, 1326 bp, Eucalyptus grandis
atggcatccatcagaaggaccttgtccccagttcctcgacctggaactccgctaaatggcgaagcctgttcagtttcatctcctttgtcgaagtcatcttcttgtgccccaaccccaagtcaaacaccatcaggtggaatactgtcaattttggtgggttcattggattctctggcttttgcactcagagctttctctcaacgaccctctaggccttacgataggcaaaaaccaagagggcaaacttggagaagggcattatttcatttcttcatttgttttgtcattgggatgttcattggagttacccaatttgcctcaatgaatgcatcgataaacttcatttcgaagaaccatgccatatcctttgagccgagcacaactttcaggaataaccagttacaagatgatgcagttagaaatgcgacaccattttacagtatgagcaatgagaatgactctaccctgcagctacaatcacatcagcagctgttgtattcgattgctcaaaatagcagtaataatgaatcagctactaaatatgcacatgtggaagttcagaagcttctgattgttgtgaccccaacacagagacacccacttcaggcctattatcttaatcgtctggcgcatacactgaagctggtaccacctcctttgttgtggatagttgtggagatgacctcacagtctccagaaacagctgacaccttgaggaagactggtatcatctatcggcatctcgtgtgcgataaaaatctgaccgaggtcacagatgtaagagttcaccaaaggaatgtggcgttatctcacattgaaacacatcatcttgatggaatagttttcttcgcagatgaggataatatctacgcaactgatctatttgatcaaatgaggcaaattaggcgctttggaacttggctggtggccaaagtgacagatagcagaagcagagctgtattggagggtcccatttgtaatggcactactgtacttgggtggcatgcaactgaatcaaggaggggattcaaaagattttatgccgacttatcaggatttgccttcaatagcacagtactatgggataccaagagatggcacagacccaccctcgaacccattaggcaatttgacacaccaaaagacaaattcccagtgagcacattagttgaacaaattgtggaggatgagagccagatggaaggcttactccaaaactgctcaagggtgatggtttggcatcttcatgtggattcatctagttacttttatccacagagttggttgacaaagagtaatctcaatgccactgcctcactgacatag

SEQ ID NO: 33 - DNA, 1224 bp, Eucalyptus grandis
atggcgtcgttcaggagaaccctgtcgccgccgtaccacgatcgccattaccagaacggcgaggccccagttccggtcccgcctccgtcgcataaacccttcgccgcctcggccaagccctcctcggcttcgccgggattcgacgtctgtggattcttgacgttcgtcttgctggggaagcagccgaggaggtcgtggaggagattgctctacaggtgtttgttgtttttccttttcgggtttctattcggcatgacccctttcgggcatgtgaacgaattgcaggctcatgacttctctttggatatcaagccgcccaatgtcaatgtcaacgctcggttggagaaggccatcgatcatccggtaaggcacgacgggtttgtgcgcgaaagttcgaatcttgaagtaggagcaggagtcagggcgaagtcagatggaagttctgatttggtgcctaggaagcagttgattgtggttacgccaacttattctagagcagcacaagcttactttttatatagattaggccaggtactgaggcttgtgccgcctccccttttgtggatagtggtggagatgaatgtcgtttccatggagactgcagatatattgaggaaaagcggagttatgtatcggcatttggtatgtacaaagaattctacagatattaaggataggggcgtgcatcagaggaacaccgcattggagcatattgagcgccataagctcgatggaattgtttactttgctgacgatgataatatctattctcttgagttgttccacagcctaagacaaattaggcgttttggcacttggccagttgccatgcttgcacagagcaaaagcaaggctattcttgaaggtccagtctgtaatggaagtcaagtgattggatggcacacaaatgagaaaagtaaaagactccgacgttttcatgttgatatgtcaggatttgcttttaacagtacaatcttgtgggatccaaagagatggaggcgcctgacgctgcatccgatacggcaattggacacagtgaaggagggttttcaggagaccacgtttatacaacaagttgtagaagatgaaagtcagatggaaggtattccttttggctgctcaagaataataaactggcatctccatttggaagctcgtaatcttatttatccaaaagggtggctatttgacaagaacctggaggccgatctaactgttacgtga

SEQ ID NO: 34 - DNA, 1239 bp, Eucalyptus grandis
atgtcaatggtgtcgagcaggagaaccttgcctccttataatgatcgcaattacccaaatggggactcctttttgttaggatcatcaccaaatcagaggccttcctatgtttttggcaaagcatcatctgcttcgctgggtttcgccgcatcaggaattagaatccgtggaatgttcgccgccattttccccggaaagtactcgagaaagtctccgccgctgtggagatcattttataggtttttgttgtttttcatactaggatttttactaggtttgatcccttatgggcatgtaaacgacttgcgagctaatcattcgttggaggttaagccgcctccgaccaatgtcaggttggatgctggagtggatcagtcgattgagcgtaaggatttcgtcattagagctgttaatttgagggtagaagaaaaggttgagatgaatttggaagagggttcaagtttggtgcctaaaaagcagctgattgtggttacaccaacgtataacagggccctgcaggcctactttttgaacaggcttggtcaggtattaaggctcgtgcaatctccattgttgtggattgtggtggagatggatgcagcttcaatggagactgcagacgtattgagaaaaaccggggtcatgtataggcatttggtgtgtgttaagaacgtgacagatgttaaagaccagggtgtgcatcagcggaacaaagcattggagcacatccagcaccatcaactcgatggcattgtttactttgcggacgactataacatttactcaatagagttgttcaagaacctaagacaaattagtcgttttggcacttggcctgtcgcgatgcttgcgcagagcaaaaacaaagtaatattggaaggtcccgtgtgcaacggaagtcaaataatgggttggcacacaaatgaaagaagtaagagactccataggttccaagttgataaatctggatttgctttcaacagtacaatcttgtgggatccaaagagatggcaccatcccatatctaatccaattcagctggtgggctctttgaaggaaggtttccagggtacaaaatttatagaacaagtggtagaagatgaaagtcaaatggaagcaattcctcctggctgctcgctaataatgaactggcatctctatttggatggtcctggtcttccgtacccccctggctggttgcttcagaagaatcttgatgatgttctccctttaagatga

SEQ ID NO: 35 - DNA, 1479 bp, Eucalyptus grandis
atgaagctcttggcaatgcagcagggccaccccgccgacggccagagcagcgccgccctccgggggctggccccgctcgactcctccaagtcgccgccggcgaccgtgttctggctggcggttcacgccctgtgctgcctcggcagcctcgtgctcggcttccgtttctctcagctgctcgtcttcctcctcttctccacgtcgccgatgtttcgtgctgccgatgaaacggcctccgacaatcctgtcctcctcctctcgaacccgagcccgacgacggtcaaccagtccttgacgcgcgtggctgtgggccggcacggaatcctggttcggccgtggccgcaccccgaccccgtcgaggtcatgaaggcgcaccggatcatagagagagtgcagagagaacagagggctcagttcagcgctaagaatcctaacccgaggtctttgattgtgatcaccccgacttacgtgcgggcattccaggcgatgcatctgacggcgctgatgcactccctgatgctcgtgccgtacgaggtggtgtggatcgtggtggaggccggcggagcgagcaacgagacggcatcgctgatcgagaggtcgggtttgaggaccgtccatgtcgggttcgagcaggagatgcccgaagcctggcaggatcggcgcagtttggaggtgaagatgagagttcatgcattgagaatcgtgagagagaagaatctggacggcattgtgatgttcgccgatgacagtaacgttcacagcatggagctctttgacaagatccaggctgtgaaatggatcggcgctgtttctttaggaatccttcctcattcgactgtggatgaatcggaccaagccgtttcgaaaatcttgaatgaaagcaattcgccacttactatccaaggtccagtttgtgattcagccaatcagctcattggctggcgcaccgataataggtcacataacatgcacaggggagcgagtcacctagatgatcagggttcgcttttgccagaaaagctggaatgggccggttttgtgctaaattctaggatgctgcggaaggatgctgaagataatccggaatgggtgaaggacttggactcactcaattgggatcaagatgttgacagtcccctgtctttgctcaaagacctatcggtgttggagccactaggaaactgcagccgccggcttatggtctggcagcttcgtgtcgaagcccgtcctggtagcgaattccctaaaggatggcttatcaaccctcccttggggatcacggtaccctcgaagcgtcctccatggcccaatgctcctccagaactcccatctatagaaaatgcaagcagcgaagaagaggagaccgaccagcacccggctgaaaatcaaaggcacgaacgaagttttgggattaagagaaggcatgagaccgagacagtcgaacccaaactgctcagaagggaacctgtagaaaagtga

SEQ ID NO: 36 - DNA, 1509 bp, Eucalyptus grandis
atgagccggcggaacaacggcttccggtggccgtcggcgccgaccgactcctcgtcggacggggccatgaagtccccggcggccgtgttctggctggtcctccacggcctctgctgcctcatcagcctcgtcctcggcttccgcttctcccgcctcgtcttcttcctcgtcttctccacctcctggaccgggccccccgccaccaccctcttcaccgcgtcgccgctccggcaggcggcggagatctccgggaatctggagactcgctcctccccggtcgccgcgggcggcggggctgcgcccgtcaacaagacggtgccccgggtcgtggtcgggcggcacgggatccggatccggccgtggccgcaccccaacccggtcgaggtcatgaaggcgcacgagatcatcgaccgggtgcagcgggagcagcgtgcccagttcggagtgaagagcccgaggacggtcatcgccgtcacgcccacctacgtcaggaccttccagacgctccacatcaccggcgtgatgcactcgctcatgctggtgccgtacgatttggtgtgggtcgtggtggaggccggcggggccagcaacgagaccgcctccatcctcgccaagtccgggctgaggaccatccacgtcgggttcgaccaagggatgccgagcacctgggcggatcggcacaagctcgaggcgaaaatgcggctgcacgcattgagaattgtgcgagaggagaagctggatggaattgtggtgtttgcggatgatagcaatatgcacagcatggagctcttcgatgagatccagaatgtgaaatggatcggcgccctttcgataggcattctggctcactcgagcacgacaacggatgaatctgagtcgtcgacggttcagcgagtggctgaagatgaaaattggccggtgcctgtgcagggtccggcctgtaactcgtctaataagctggtcggctggcacaccttcaatacgcagccttatgaggggaaggctgcgatttacatagacgatcgagcgacagtgttgccacggaagttagagtggtcaggattcgtgctgaactctaggctggtttggaaggatgcggaggataaaccggagtgggcgaacgatctggattcgctcgagggggttgatgatgtcgagagtcccctgtcccttgcgaaagacccttctgttgtggagcctcttggaagctgtggccgccaagtcatgctctggtggcttcgtgttgaagctcgttccgacagcagatttcctcctggatgggtaattgaccctcctttggagatcactgtcccagcaaaacgcacaccatggcccgatgctcctccagagcttccatccactgaaaaagtgatgaacatccaagataaccaagtggtgaagcgtccaacaaaaactcgatcgtccagatcgaagcgaagttctcggagtaagaggaagcgtgaggcaagaatagcagagacagatatccgaggaaagcataccgattaa

SEQ ID NO: 37 - DNA, 1008 bp, Eucalyptus grandis
atgggatctgtggaaagatcaaggaagaaatcacagcgttggaagagagccattttccgtttctctgtatgcctcgttattggtttcttcacaggcttcgctcagacaattaaagcctccagttttgccggtcgtgtagcagttacctcggataaagcagatttctatcctcagacagcgccaaaaccgaggataagcatcgtcgatgcgagttcaaaagctgagacaccggccgtggcagctttggctggaggagaggctcagttacagcaagaagaagaggaagtcaaattgattccaagaagacttctcatatttgtcacgcccaccgggacaaaacataagtttcaaggaatcttcctaagaaggttgtccaacactttgagacttattcctgcacctttgctctggatcattgtggagggacaatccgaatccaaagaagtatcggatatactcaggaacactggcattatgtataggcatttggtctccaaggagaacttcactgacccgcaagcaggaacgaatcatcagttcaacgttgctctgcagcacatcgtgcaccaccggataagtgggatcctccatctttcgatgatttcgaatgcttatgatttggatttctttgaggggctcagggagatagaggtgttgggaaagtggccagtgtgcaaacttgcttcacaagttagaggatggcatgatggtgacaagagcaatgacacagacccaagacctccattgatgatgattcatccttcaagttttgcattcaacagttcgattctgtgggactctgaaagatggggtcgatcttcgtcgtctgctcaacctaccaccaccacgaacaatgccggccaggatctattgaagtttgtgaagcaaattgttattgaggatgacacgaatctgaaaggcgttcaaggaaaagattgctccaagaacatggtttggcatcaggaactccagattcattctgggatgtcaaaaatcgaacagttatga

SEQ ID NO: 38 - protein, 376 aa, Eucalyptus grandis
MGSVERSKKKVQLWKKAIVHFSLCFVMGFFTGFAPTGKARTFAGQPVMSNKIEIFSPQPGELSDPPPTTSSHVSFNRSLISEVPERVSGKPRAEPEEDDLLENEDNQLEEAEFPEGNNKLVPRRLLIIVTPTTNHPLTGVLLRRLANTIKLVPQPLLWIIVEKRSDSREVSEMMRKTGIMYRHLVFRENFTDPDAEMDHQRNLALRHIEHHKLSGIVHFAGLSNVYDLDFFQELREIEVFGTWPMAVLSANRKKVNIEGPVCDSSQVLGWHLRRLSNETTATSESSERPPIHISSFAFNSSILWDPERWGRPSSGQPNSQDSMKFVKEVVHEDETKVKGLPGEKCSKIMLWHLRYLLPRPHHPPSTALHQTLTSQR

SEQ ID NO: 39 - protein, 441 aa, Eucalyptus grandis
MASIRRTLSPVPRPGTPLNGEACSVSSPLSKSSSCAPTPSQTPSGGILSILVGSLDSLAFALRAFSQRPSRPYDRQKPRGQTWRRALFHFFICFVIGMFIGVTQFASMNASINFISKNHAISFEPSTTFRNNQLQDDAVRNATPFYSMSNENDSTLQLQSHQQLLYSIAQNSSNNESATKYAHVEVQKLLIVVTPTQRHPLQAYYLNRLAHTLKLVPPPLLWIVVEMTSQSPETADTLRKTGIIYRHLVCDKNLTEVTDVRVHQRNVALSHIETHHLDGIVFFADEDNIYATDLFDQMRQIRRFGTWLVAKVTDSRSRAVLEGPICNGTTVLGWHATESRRGFKRFYADLSGFAFNSTVLWDTKRWHRPTLEPIRQFDTPKDKFPVSTLVEQIVEDESQMEGLLQNCSRVMVWHLHVDSSSYFYPQSWLTKSNLNATASLT

SEQ ID NO: 40 - protein, 407 aa, Eucalyptus grandis
MASFRRTLSPPYHDRHYQNGEAPVPVPPPSHKPFAASAKPSSASPGFDVCGFLTFVLLGKQPRRSWRRLLYRCLLFFLFGFLFGMTPFGHVNELQAHDFSLDIKPPNVNVNARLEKAIDHPVRHDGFVRESSNLEVGAGVRAKSDGSSDLVPRKQLIVVTPTYSRAAQAYFLYRLGQVLRLVPPPLLWIVVEMNVVSMETADILRKSGVMYRHLVCTKNSTDIKDRGVHQRNTALEHIERHKLDGIVYFADDDNIYSLELFHSLRQIRRFGTWPVAMLAQSKSKAILEGPVCNGSQVIGWHTNEKSKRLRRFHVDMSGFAFNSTILWDPKRWRRLTLHPIRQLDTVKEGFQETTFIQQVVEDESQMEGIPFGCSRIINWHLHLEARNLIYPKGWLFDKNLEADLTVT

SEQ ID NO: 41 - protein, 412 aa, Eucalyptus grandis
MSMVSSRRTLPPYNDRNYPNGDSFLLGSSPNQRPSYVFGKASSASLGFAASGIRIRGMFAAIFPGKYSRKSPPLWRSFYRFLLFFILGFLLGLIPYGHVNDLRANHSLEVKPPPTNVRLDAGVDQSIERKDFVIRAVNLRVEEKVEMNLEEGSSLVPKKQLIVVTPTYNRALQAYFLNRLGQVLRLVQSPLLWIVVEMDAASMETADVLRKTGVMYRHLVCVKNVTDVKDQGVHQRNKALEHIQHHQLDGIVYFADDYNIYSIELFKNLRQISRFGTWPVAMLAQSKNKVILEGPVCNGSQIMGWHTNERSKRLHRFQVDKSGFAFNSTILWDPKRWHHPISNPIQLVGSLKEGFQGTKFIEQVVEDESQMEAIPPGCSLIMNWHLYLDGPGLPYPPGWLLQKNLDDVLPLR

SEQ ID NO: 42 - protein, 492 aa, Eucalyptus grandis
MKLLAMQQGHPADGQSSAALRGLAPLDSSKSPPATVFWLAVHALCCLGSLVLGFRFSQLLVFLLFSTSPMFRAADETASDNPVLLLSNPSPTTVNQSLTRVAVGRHGILVRPWPHPDPVEVMKAHRIIERVQREQRAQFSAKNPNPRSLIVITPTYVRAFQAMHLTALMHSLMLVPYEVVWIVVEAGGASNETASLIERSGLRTVHVGFEQEMPEAWQDRRSLEVKMRVHALRIVREKNLDGIVMFADDSNVHSMELFDKIQAVKWIGAVSLGILPHSTVDESDQAVSKILNESNSPLTIQGPVCDSANQLIGWRTDNRSHNMHRGASHLDDQGSLLPEKLEWAGFVLNSRMLRKDAEDNPEWVKDLDSLNWDQDVDSPLSLLKDLSVLEPLGNCSRRLMVWQLRVEARPGSEFPKGWLINPPLGITVPSKRPPWPNAPPELPSIENASSEEEETDQHPAENQRHERSFGIKRRHETETVEPKLLRREPVEK

SEQ ID NO: 43 - protein, 502 aa, Eucalyptus grandis
MSRRNNGFRWPSAPTDSSSDGAMKSPAAVFWLVLHGLCCLISLVLGFRFSRLVFFLVFSTSWTGPPATTLFTASPLRQAAEISGNLETRSSPVAAGGGAAPVNKTVPRVVVGRHGIRIRPWPHPNPVEVMKAHEIIDRVQREQRAQFGVKSPRTVIAVTPTYVRTFQTLHITGVMHSLMLVPYDLVWVVVEAGGASNETASILAKSGLRTIHVGFDQGMPSTWADRHKLEAKMRLHALRIVREEKLDGIVVFADDSNMHSMELFDEIQNVKWIGALSIGILAHSSTTTDESESSTVQRVAEDENWPVPVQGPACNSSNKLVGWHTFNTQPYEGKAAIYIDDRATVLPRKLEWSGFVLNSRLVWKDAEDKPEWANDLDSLEGVDDVESPLSLAKDPSVVEPLGSCGRQVMLWWLRVEARSDSRFPPGWVIDPPLEITVPAKRTPWPDAPPELPSTEKVMNIQDNQVVKRPTKTRSSRSKRSSRSKRKREARIAETDIRGKHTD

SEQ ID NO: 44 - protein, 335 aa, Eucalyptus grandis
MGSVERSRKKSQRWKRAIFRFSVCLVIGFFTGFAQTIKASSFAGRVAVTSDKADFYPQTAPKPRISIVDASSKAETPAVAALAGGEAQLQQEEEEVKLIPRRLLIFVTPTGTKHKFQGIFLRRLSNTLRLIPAPLLWIIVEGQSESKEVSDILRNTGIMYRHLVSKENFTDPQAGTNHQFNVALQHIVHHRISGILHLSMISNAYDLDFFEGLREIEVLGKWPVCKLASQVRGWHDGDKSNDTDPRPPLMMIHPSSFAFNSSILWDSERWGRSSSSAQPTTTTNNAGQDLLKFVKQIVIEDDTNLKGVQGKDCSKNMVWHQELQIHSGMSKIEQL